(19) United States
(12) Patent Application Publication
(10) Pub. No.: US 2003/0103988 A1
(43) Pub. Date: Jun. 5, 2003

(54) REGULATION OF ACTIVATED T CELLS BY RECOGNITION OF T CELL RECEPTOR BETA CHAINS AND MAJOR HISTOCOMPATIBILITY COMPLEX CLASS IB MOLECULES

(76) Inventors: LEONARD CHESS, SCARSDALE, NY (US); RANDLE WARE, NEW YORK, NY (US); HONG JIANG, NEW YORK, NY (US); BENVENUTO PERNIS, BRONXVILLE, NY (US)

Correspondence Address: JOHN P WHITE, COOPER & DUNHAM, 1185 AVENUE OF THE AMERICAS, NEW YORK, NY, 10036

(21) Appl. No.: 08/548,595

(22) Filed: Oct. 26, 1995

(51) Int. Cl. A61K 39/395; A61K 39/38; A01N 1/02; A61K 39/00

(52) U.S. Cl. 424/184.1; 424/810; 435/2

(57) ABSTRACT

The level of CD8⁺ T cell cytotoxicity directed toward activated CD4⁺ T cells expressing a specific T cell receptor Vβ chain and a major histocompatibility complex class Ib molecule is assayed by contacting a sample containing CD8⁺ T cells with the activated CD4⁺ T cells for a determined period of time and determining the amount of activated CD4⁺ T cell death during the time period. The level of CD8⁺ T cell activity stimulated by the activated CD4⁺ T cells is assayed by measuring lymphokine release from stimulated CD8⁺ T cells or by determining the amount of cell surface molecules specifically expressed on stimulated CD8⁺ T cells. An agent capable of stimulating or inhibiting CD8⁺ T cells cytotoxicity toward the activated CD4⁺ T cells will suppress or inhibit the suppression of an immune response mediated by the activated CD4⁺ T cells, respectively.

The invention disclosed herein was made with Government support under NIH Grant Nos. AI 14969 and AI 24748 from the Department of Health and Human Services. Accordingly, the U.S. Government has certain rights in this invention.

Throughout this application, various references are referred to within parentheses. Disclosures of these publications in their entireties are hereby incorporated by reference into this application to more fully describe the state of the art to which this invention pertains. Full bibliographic citation for these references may be found at the end of this application, preceding the claims.

### BACKGROUND OF THE INVENTION

A variety of studies have provided evidence that CD8⁺ T cells interact with CD4⁺ T cells to regulate immune responses (Bloom et al., 1992a; Eardley et al., 1978; Jandinski et al., 1976; Thomas et al., 1980). It was speculated that these regulatory interactions between CD4⁺ and CD8⁺ T cells are complex and may involve both antigen specific as well as non-specific mechanisms. In principle, one can envision three distinct, but not mutually exclusive, models by which CD8⁺ T cells could specifically regulate antigen driven CD4⁺ T cells. In the first model both CD4⁺ and CD8⁺ T cells may recognize antigen-MHC complexes on conventional antigen presenting cells. Because of the proximity of the CD8⁺ cells to the CD4⁺ cells, the CD8⁺ T cells could release lymphokines that regulate CD4⁺ T cell function (Bloom et al., 1992b; Salgame et al., 1991). In the second model, antigen-MHC class II complexes on conventional antigen presenting cells induce CD4⁺ T cells to acquire a new cell surface phenotype defined by the expression of non-polymorphic membrane molecules unrelated either to antigen or to the T cell receptor (TCR), which can be recognized by regulatory CD8⁺ T cells. Because CD4⁺ T cells are known to at least transiently express activation antigens, these molecules could be used by CD8⁺ T cells to regulate immune response. In the third model, T cell receptor related structures such as TCR-derived peptide/MHC complexes expressed on antigen activated CD4⁺ cells induce CD8⁺ regulatory cells. In this case the TCR structures would be predicted to bind and be recognized in the context of MHC class I molecules. These CD8 cells differentiate and recognize the TCR-derived peptide/MHC class I complexes expressed on the activated CD4⁺ inducer cells. The effector phase of regulation mediated by these putative TCR peptide recognizing CD8⁺ T cells could either be by direct cytolysis and/or the release of cytokines. This view of immune regulation was initially suggested, in principle, by Jerne (Jerne, 1975) in his idiotypic hypothesis, and many of the proposed suppressor cell interaction models proposed during the latter part of the 1970's and early 1980's involved recognition of TCR structures (Dorf and Benacerraf, 1984; Goodman and Sercarz, 1983; Green et al., 1983).

In this regard, recent experiments in animals strongly implicate the TCR variable chains expressed by CD4⁺ cells as being responsible for recognition by regulatory CD8⁺ T cells. Injection of mice with superantigen is followed by an initial expansion of the T cells characteristic of the superantigen. This is followed by selective elimination and/or induction of anergy in these cells (Kawabe and Ochi, 1991; McCormack et al., 1993; Rellahan et al., 1990; Webb et al., 1990). For example, after intravenous injection of the superantigen staphylococcus enterotoxin B (SEB), there is an initial deletion (12-24 hours) followed by an expansion and a second phase of deletion (after day 4) of CD4⁺ and CD8⁺ T cells that express T cell receptor (TCR) Vβ8 chains (Gonzalo et al., 1994; Kawabe and Ochi, 1991; Rellahan et al., 1990). The increase in CD4⁺Vβ8⁺ and CD8⁺Vβ8⁺ T cells reaches a maximum on day 4 and by day 8 returns to background. However, the CD4⁺Vβ8⁺ but not the CD8⁺Vβ8⁺ T cell population is further deleted, becomes reduced to about 30-40% below baseline and remains at this reduced level for at least 21 days. The mechanism of this delayed deletion of CD4⁺ T cells following SEB administration is unknown. Although it is known that triggering of the TCR alone by either superantigen or anti-TCR antibodies can induce apoptosis (Takahashi et al., 1989; Lenardo, 1991; Boehme and Lenardo, 1993), other mechanisms requiring interactions with other immunoregulatory cells may also contribute to the deletion of CD4⁺ T cells. The mechanism of CD4⁺ T cell deletion in this context is of interest because it might shed light on the mechanisms of T lymphocyte regulation in general.

Although various mechanisms for the elimination of the responding T cells have been suggested, recent studies indicate that CD8⁺ T cells are at least partially involved. Specifically in experimental allergic encephalomyelitis (EAE), an autoimmune disease induced by CD4⁺ T cells which predominately utilize Vβ8.2 T cell receptor molecules (Koh et al., 1992), animals which recover spontaneously are resistant to relapses and to a second induction of disease only if they possess CD8⁺ T cells (Jiang et al., 1992; Koh et al., 1992). In addition, vaccination of rats (Howell et al., 1989; Vandenbark et al., 1989) and of mice (Gaur, et al, 1992) with a peptide representing a portion of Vβ8.2 also confers resistance to EAE. More generally, TCR-peptide vaccination of mice leads to anergy in the corresponding set of CD4⁺ cells only if CD8⁺ cells are present (Gaur et al., 1993). These data suggest the possibility that CD8⁺ T cells may regulate CD4⁺ T cells at least in part on the basis of CD4⁺ T cell TCR Vβ chain usage.

Previous studies in the human immune system have also provided evidence that recognition of the TCR is integral to the specificity of immune regulation between T cells subsets in vitro. For example, a series of experiments have shown that it is possible to generate human CD4⁺ T cell clones which proliferate specifically to autologous CD4⁺ clones that either show a particular antigen specificity or specific Vβ expression (Lamb and Feldman; 1982, Naor et al., 1991). In other experiments human CD8⁺ T cell clones raised to autologous allo-reactive CD4⁺ cell lines have been shown to inhibit fresh autologous CD4⁺ T cells from proliferating to the same alloantigen (Koide and Engleman, 1990). These results were interpreted as being consistent with the idea that T-T cell interactions may involve all or part of the TCR as a target of recognition.

### SUMMARY OF THE INVENTION

The level of CD8⁺ T cell cytotoxicity directed toward activated CD4⁺ T cells expressing a specific T cell receptor Vβ chain and a major histocompatibility complex class Ib molecule is assayed by contacting a sample containing CD8⁺ T cells with the activated CD4⁺ T cells for a determined period of time and determining the amount of activated CD4⁺ T cell death during the time period. The level of CD8⁺ T cell activity stimulated by the activated CD4⁺ T cells is assayed by measuring lymphokine release from stimulated CD8⁺ T cells or by determining the amount of cell surface molecules specifically expressed on stimulated CD8⁺ T cells. An agent capable of stimulating or inhibiting CD8⁺ T cells cytotoxicity toward the activated CD4⁺ T cells will suppress or inhibit the suppression of an immune response mediated by the activated CD4⁺ T cells, respectively.

### BRIEF DESCRIPTION OF THE FIGURES

FIGS. 1A, 1B, and 1C show the cytotoxicity effect of CD8⁺ T cell clones against autologous Vβ2⁺ CD4⁺ T cells. The relative cytotoxicity of anti-JK50t clones against the inducing clone JKSOt as compared to the autologous lymphoblastoid line is shown (A). For each clone designated on the horizontal axis the vertical axis represents the percent specific cytotoxicity to the inducing clone JK50t divided by the percent specific cytotoxicity to the lymphoblastoid line (A). A subclone of JK4/2 was used as an effector against randomly selected Vβ2⁺ CD4⁺ and Vβ2⁻ CD4⁺ clones in a 14 hour chromium release assay (B). The same subclone and the CD8⁺ Vβ2⁺ clone JK214t were used as effectors against the inducing clone JK50t and the Vβ2⁻CD4⁺ tetanus-toxoid responsive line JK(TT) in a 14 hour chromium release assay in the presence and absence of TSST-1 (100 ng/ml) (C). The result presented is the average of 2 independent experiments (C).

FIGS. 2A and 2B show the specificity of a polyclonal CD8⁺ anti-JK50t line. Four Vβ2⁺ CD4⁺ lines (A) and three Vβ2^{- CD}4⁺ lines (B) were used as targets in a 14 hour Cr51-release assay with graded numbers of the anti-JK50t line as effector. “t” and “s” designate Vβ2⁺ and Vβ2 clones respectively; JK{SEB} is an autologous CD4⁺ line raised to SEB (Vβ2⁻); JK{TSST} is a CD4⁺ line raised to TSST-1 (50% Vβ2⁺).

FIG. 3 shows cold target inhibition of cytotoxicity by the anti-JK50t line to its inducing clone. Cold target inhibition was performed by using an assay mixture of the CD8⁺ anti-JK50t line as effector (10⁵ per well) and clone JK50t as labeled target (2×10⁴ per well). Baseline killing at a 5:1 E:T ratio was 20%, as shown in FIG. 2. The polyclonal Vβ2+ and Vβ2⁻ lines shown in FIG. 2 or clone JK50t were added as cold target inhibitors at the indicated cold:labeled ratio and the percent inhibition of cytotoxic release at 14 hours determined as described in the Methods section.

FIG. 4 shows a time course of sensitivity to cytolytic action. The Vβ2⁺ CD4⁺ and Vβ2⁻ CD4⁺ clones JK50t and JK202s respectively were used as targets in a 14 hour chromium release assay with the anti-JK50t line over two cycles of stimulation. Arrows indicate times of stimulation with phorbol/ionomycin. In parallel control experiments the addition of Con A (25 micrograms/ml) resulted in killing of all clones at all times (data not shown).

FIGS. 5A and 5B show comparative cytotoxicity of CD8⁺ clones expanded on TC3/1 (A) and TC2/151 (B). Anti-T cell clones were prepared by plating CD4⁺-depleted PBL from donor TC in 96 well U-bottom plates on the appropriate feeders as described in Methods. Wells that grew sufficiently for testing were used as effectors in a 14 hour ⁵¹Cr release assay against the inducing Vβ2⁺ (TC2/151) and Vβ3⁺ (TC3/1) clones.

FIG. 6 shows the specific cytotoxicity of the CD8⁺ clone TC12/7. The CD8⁺ clone TC12/7 shown in the screening in FIGS. 5A and 5B was tested for cytotoxicity in a 14 hour ⁵¹Cr release assay against its inducing CD4⁺ clone TC2/151, 2 independently isolated Vβ2⁺ CD4⁺ clones TC2/9 and TC2/109, and the Vβ3⁺ CD4⁺ clone TC3/1.

FIGS. 7A and 7B show the effect of SEB on CD4⁺Vβ8.1,2⁺ (A) and CD4⁺ Vβ6⁺ (B) populations of T cells in normal mice and in mice depleted of CD8⁺ cells. CD8⁺ T cell depletion protects CD4⁺Vβ8.1,2⁺ T cells from SEB induced T cell death. The experiments were done in BALB/c mice and as described in Material and methods. The group designations are: CD8⁺/PBS, PBS-primed, CD8⁺ T cell non-depleted; CD8⁺/PBS, PBS-primed, CD8⁺ T cell depleted; CD8⁺/SEB, SEB-primed, CD8⁺ T cell non-depleted; CD8⁺/SEB, SEB-primed, CD8⁺ T cell depleted. The values for day 1 is the average of >50 normal mice. For days 4, 7, 14 and 21, each point represents the average of the data of 12-18 mice from three separate experiments

FIGS. 8A and 8B show FACS profiles of data from Table 4, in FACS assay, at E/T ratio of 2.5:1. (A) shows the profile of control group (without L3), the ratio of specific targets/non-specific targets was 2.0; (B) shows the profile of experimental group (with L3), the ratio of specific targets/non-specific targets was 0.70, therefore, the specific CTL activity of L3 cells was calculated as: (2.0−0.7)/2.0=65%.

FIG. 9 shows the killing effect of TCR-Vβ8 specific CD8⁺ T cells on CD4⁺Vβ8⁺ target T cells but not on CD4⁺Vβ8⁻ target T cells. ⁵¹Cr-release assay was used to detect the killing capacity of TCR Vβ8 specific CD8⁺Vβ8⁻ T cell lines. SEB activated CD4⁺Vβ8⁺ T cells and SEE activated CD4⁺Vβ8⁻ T cells (prepared as described in Experimental Details) were used as specific and non-specific targets. This figure represents data from three separate experiments with four independent CTL lines.

### DETAILED DESCRIPTION OF THE INVENTION

The present invention provides a method for assaying the level of CD8⁺ T cell cytotoxicity directed toward activated CD4⁺ T cells expressing a specific T cell receptor Vβ chain and a major histocompatibility complex class Ib molecule in a sample, comprising:

a) contacting the sample with the activated CD4⁺ T cells expressing the specific T cell receptor Vβ chain and the major histocompatibility complex class Ib molecule for a determined period of time; and

b) determining the amount of activated CD4⁺ T cell death during the time period, thereby assaying the level of CD8⁺ T cell cytotoxicity directed toward activated CD4⁺ T cells expressing the specific T cell receptor Vβ chain and the major histocompatibility complex class Ib molecule.

In one embodiment of the invention, the major histocompatibility complex molecule is murine Qa-1b or a non-murine class 1b molecule homologous to murine Qa-1b.

The term “homologous” is used herein to designate a similarity in structure and function of two molecules in two different species due to common evolutionary origin.

In another embodiment of the invention, the sample is a biological sample derived from a subject. The biological sample can be serum or a tissue sample and the subject can be a mammal such as a human or a mouse.

In yet another embodiment of the invention, the activated CD4⁺ T cells are labeled with ⁵¹Cr and the amount of activated CD4⁺ T cell death is determined by measuring the amount of ⁵¹Cr released from the ⁵¹Cr-labeled activated CD4⁺ T cells.

In another embodiment of the invention, the activated CD4⁺ T cells are labeled with a fluorescent agent and the amount of activated CD4⁺ T cell death is determined by measuring the number of the fluorescently labeled and live activated CD4⁺ T cells by fluorescence associated cell sorter (FACS) analysis.

The present invention further provides a method for assaying the level of CD8⁺ T cell lymphokine-secreting activity stimulated by activated CD4⁺ T cells expressing a specific T cell receptor Vβ chain and a major histocompatibility complex class Ib molecule in a sample, comprising:

a) contacting the sample with the activated CD4⁺ T cells expressing the specific T cell receptor Vβ chain and the major histocompatibility complex class Ib molecule for a determined period of time to stimulate CD8⁺ T cells present in the sample; and

b) determining the amount of a lymphokine released by stimulated CD8⁺ T cells during the time period, thereby assaying the level of CD8⁺ T cell lymphokine-secreting activity stimulated by activated CD4⁺ T cells expressing the specific T cell receptor Vβ chain and the major histocompatibility complex class Ib molecule.

In one embodiment of the invention, the major histocompatibility complex molecule is murine Qa-1b or a non-murine class 1b molecule homologous to murine Qa-1b.

In another embodiment of the invention, the sample is a biological sample derived from a subject. The biological sample can be serum or a tissue sample and the subject can be a mammal such as a human or a mouse.

In yet another embodiment of the invention, the lymphokine is selected from the group consisting of interleukin-2, γ interferon, and tumor growth factor-beta (TGF-β).

In another embodiment of the invention, the amount of lymphokine is determined by radioimmunoassay (RIA), enzyme-linked immunosorbent assay (ELISA), specific protein mass assay, or activity assay.

The present invention also provides a method for assaying the level of CD8⁺ T cell activity stimulated by activated CD4⁺ T cells expressing a specific T cell receptor Vβ chain and a major histocompatibility complex class Ib molecule in a sample, comprising:

a) contacting the sample with the activated CD4⁺ T cells expressing the specific T cell receptor Vβ chain and the major histocompatibility complex class Ib molecule for a determined period of time to stimulate CD8⁺ T cells present in the sample; and

b) determining the amount of a cell surface molecule specifically expressed on stimulated CD8⁺ T cells, thereby assaying the level of CD8⁺ T cell activity stimulated by activated CD4⁺ T cells expressing a specific T cell receptor Vβ chain and a major histocompatibility complex class Ib molecule.

In one embodiment of the invention, the major histocompatibility complex molecule is murine Qa-1b or a non-murine class 1b molecule homologous to murine Qa-1b.

In another embodiment of the invention, the sample is a biological sample derived from a subject. The biological sample can be serum or a tissue sample and the subject can be a mammal such as a human or a mouse.

In yet another embodiment of the invention, the cell surface molecule specifically expressed on stimulated CD8⁺ T cells is an interleukin-2 receptor.

In another embodiment of the invention, the cell surface molecule specifically expressed on stimulated CD8⁺ T cells is a receptor that recognizes a complex of the major histocompatibility complex class Ib molecule and the Vβ chain or a complex of the binding domains of the major histocompatibility complex class Ib molecule and the Vβ chain.

In yet another embodiment of the invention, the cell surface molecule specifically expressed on stimulated CD8⁺ T cells is labeled with a fluorescent agent and the amount of the cell surface receptor is determined by measuring the intensity of the fluorescently labeled CD8⁺ T cells by fluorescence associated cell sorter (FACS) analysis.

The present invention further provides a method of suppressing an immune response mediated by activated CD4⁺ T cells expressing a specific T cell receptor Vβ chain and a major histocompatibility complex class Ib molecule in a subject comprising administering to the subject an effective amount of an agent capable of stimulating CD8⁺ T cell cytotoxicity directed specifically toward the activated CD4⁺ T cells, thereby suppressing the immune response in the subject.

In one embodiment of the invention, the agent is a cell that expresses on the cell surface the specific T cell receptor Vβ chain and the major histocompatibility complex class Ib molecule or a chimeric Vβ-MHC class Ib molecule. Expression vectors with nucleic acids encoding these molecules can be used to produce this cell.

In another embodiment of the invention, the agent comprises the major histocompatibility complex class Ib molecule or a CD8⁺ T cell-binding domain thereof complexed to the Vβ chain or a CD8⁺ T cell-binding domain thereof.

In yet another embodiment of the invention, the agent is administered orally, subcutaneously, or intravenously.

In another embodiment of the invention, the major histocompatibility complex molecule is murine Qa-1b or a non-murine class 1b molecule homologous to murine Qa-1b.

In yet another embodiment of the invention, the method of suppressing an immune response is used in treating an autoimmune disease. The autoimmune disease is selected from the group consisting of rheumatoid arthritis, multiple sclerosis, scleroderma, systemic lupus erythematosus, idiopathic thrombocytopenia purpura, hemolytic anemia, diabetes, and juvenile diabetes.

The present invention also provides a method of suppressing an immune response mediated by activated CD4⁺ T cells expressing a specific T cell receptor Vβ chain and a major histocompatibility complex class Ib molecule, in a subject comprising:

a) contacting CD8⁺ T cells with an effective amount of an agent capable of stimulating CD8⁺ T cell cytotoxicity directed specifically toward activated CD4⁺ T cells expressing the specific T cell receptor Vβ chain and the major histocompatibility complex class Ib molecule; and

b) administering to the subject an amount of the stimulated CD8⁺ T cells effective to kill the activated CD4⁺ T cells, thereby suppressing the immune response in the subject.

In one embodiment, the agent is a cell that expresses on the cell surface the specific T cell receptor Vβ chain and the major histocompatibility complex class Ib molecule.

In another embodiment, the agent comprises a complex of the major histocompatibility complex class Ib molecule and the Vβ chain or a complex of the binding domains of the major histocompatibility complex class Ib molecule and the Vβ chain that are recognized by CD8⁺ T cells.

In yet another embodiment, the stimulated CD8⁺ T cells are administered intravenously.

In another embodiment of the invention, the major histocompatibility complex molecule is murine Qa-1b or a non-murine class 1b molecule homologous to murine Qa-1b.

In yet another embodiment, the method of suppressing an immune response is used in treating an autoimmune disease. The autoimmune disease is selected from the group consisting of rheumatoid arthritis, multiple sclerosis, scleroderma, systemic lupus erythematosus, idiopathic thrombocytopenia purpura, hemolytic anemia, diabetes, and juvenile diabetes.

The present invention further provides a method of suppressing an immune response mediated by activated CD4⁺ T cells expressing a specific T cell receptor Vβ chain and a major histocompatibility complex class Ib molecule in a subject, comprising: administering to the subject an effective amount of an agent capable of inducing expression of the major histocompatibility complex class Ib molecule on the surface of cells that express the T cell receptor Vβ chain, so as to stimulate CD8⁺ T cell cytotoxicity directed specifically toward the activated CD4⁺ T cells thereby suppressing the immune response in the subject.

In one embodiment of the invention, the agent is selected from the group consisting of cytokines, interferons such as β interferon, and heat shock proteins.

In another embodiment of the invention, the agent is administered orally, subcutaneously, or intravenously.

In yet another embodiment of the invention, the major histocompatibility complex molecule is murine Qa-1b or a non-murine class 1b molecule homologous to murine Qa-1b.

In another embodiment of the invention, the method of suppressing an immune response is used in treating an autoimmune disease. The autoimmune disease is selected from the group consisting of rheumatoid arthritis, multiple sclerosis, scleroderma, systemic lupus erythematosus, idiopathic thrombocytopenia purpura, hemolytic anemia, diabetes, and juvenile diabetes.

The present invention also provides a method of inhibiting the suppression of an immune response mediated by activated CD4⁺ T cells expressing a specific T cell receptor Vβ chain and a major histocompatibility complex class Ib molecule in a subject, comprising: administering to the subject an effective amount of an agent capable of inhibiting the stimulation of CD8⁺ T cell cytotoxicity directed specifically toward the activated CD4⁺ cells by the major histocompatibility complex class Ib molecule and the T cell receptor Vβ chain on the cell surface of activated CD4⁺ cells, thereby inhibiting the suppression of the immune response in the subject.

In one embodiment of the invention, the agent is administered orally, subcutaneously, or intravenously.

In another embodiment of the invention, the agent is an antibody capable of specifically binding to the major histocompatibility complex molecule or the specific T cell receptor Vβ chain. The agent can also be an antibody capable of specifically binding to a complex of the major histocompatibility complex class Ib molecule and the Vβ chain or a complex of the binding domains of the major histocompatibility complex class Ib molecule and the Vβ chain that are recognized by CD8⁺ T cells.

In yet another embodiment of the invention, the major histocompatibility complex molecule is murine Qa-1b or a non-murine class 1b molecule homologous to murine Qa-1b.

In another embodiment of the invention, the subject can be a mammal such as a human or a mouse.

In yet another embodiment of the invention, the method of inhibiting the suppression of an immune response is used in treating a disease selected from the group consisting of acquired immunodeficiency syndrome, chronic tuberculosis, chronic leprosy, and chronic tumors.

This invention will be better understood from the Experimental Details which follow. However, one skilled in the art will readily appreciate that the specific methods and results discussed are merely illustrative of the invention as described more fully in the claims which follow thereafter.

Experimental Details

### EXAMPLE 1

In order to develop a system to more directly study the role of TCR structures in the interactions of CD8⁺ cells with CD4⁺ cells in regulating immune responses clones of CD4⁺ cells were isolated expressing identified TCR Vβ chains. This was accomplished by stimulating purified populations of CD4⁺ cells with either superantigens or monoclonal antibodies to known TCR VP families. The resultant CD4⁺ cells expressing a given Vβ TCR were then used as immunogens to stimulate purified populations of autologous CD8⁺ cells. CD8⁺ lines and clones obtained in this manner were found to specifically recognize and lyse the inducing CD4⁺ clone but not autologous clones or lines expressing different TCR Vβ's. Further analysis of the specificity of these CD8⁺ cells demonstrated that they also recognize and kill, to a lesser degree, independently isolated autologous CD4⁺ T cell clones and lines expressing the same TCR Vβ as the inducing clone. This Vβ specific cytotoxicity was not blocked by the monoclonal antibody W6/32, which reacts with non polymorphic determinants present on HLA Class I A/B/C molecules. These results demonstrate that there are CD8⁺ T cells present in peripheral blood that interact with CD4⁺ T cells based on CD4⁺ TCR VP usage. Cells of this type have the capacity to regulate immune responses by directly killing antigen activated CD4⁺ inducer clones.

Experimental Procedures

Isolation of Lymhocyte Subsets and CD4⁺ Clones

Peripheral blood lymphocytes (PBL) were isolated as described (Friedman et al., 1981). Briefly, PBL were isolated from healthy donors by sedimentation of heparinized blood over Histopaque (Sigma Chemical Company, St. Louis, Mo.). CD8⁺ cell fractions were prepared by incubating 10×10⁶ freshly isolated PBL with 15×10⁶ anti-CD4-coated magnetic beads for 30 minutes at room temperature. Beads and adherent CD4⁺ cells were removed by magnetic separation (Dynal, Inc., Lake Success, N.Y.). Depletion was monitored by cytofluorographic analysis and repeated if CD4⁺ cells represented greater than 1% of the remaining population. SEB and TSST-1 responsive clones were isolated by limiting dilution plating of freshly isolated PBL added to irradiated (2000 rad) autologous PBL (10⁵ cells/well) in 96 well U-bottom plates (Nunc Inc., Naperville, Ill.) in medium consisting of IMDM, 10% autologous serum, 1% penicillin-streptomycin, and 100 ng/ml toxin (Toxin Technology, Madison, Wis.). Isolation of CD4⁺ clones by FACS-sorting was accomplished as described (Dangl et al., 1982). Freshly isolated PBL were stained with saturating amounts of monoclonal antibodies to CD8 (FITC-conjugated) and to either TCR Vβ2 or TCR Vβ3 (biotin-conjugated) and separated on a fluorescence activated cell sorter (Becton Dickinson, San Jose, Calif.). Cells were gated to select CD8⁻ cells of a defined Vβ phenotype and sorted into 96 well U-bottom plates containing 10⁵ irradiated autologous PBL/well in 0.1 ml of medium. Independent of the method of cloning, IL-2 (Chiron Corp., Emeryville, Calif.) was added after 6 days of culture, and after 10-14 days proliferating wells were screened for CD4⁺ and TCR Vβ usage. Selected wells were subcloned at limiting dilution by stimulation with superantigen or OKT3 and maintained subsequently by weekly stimulation using phorbol dibutyrate (20 ng/ml) and ionomycin (0.4 micromolar, 6 hr at 37° C., both from Sigma Chemicals).

Generation of Autologous CD8⁺ Anti-CD4⁺ T Cell Clones and Lines

CD8⁺ lines directed against chosen CD4⁺ clones were raised by incubating 10×10⁶ freshly isolated CD4⁺-depleted PBL and 5×10⁶ irradiated (2000 rad) CD4⁺ inducer clones in 50 cc flasks (Costar Corp., Cambridge, Mass.) in 15 cc of medium consisting of IMDM, 10% autologous serum, and 1% penicillin-streptomycin. CD8⁺ clones against chosen CD4⁺ T cell clones were raised by incubating 5×10⁴ freshly isolated CD4⁺ T cell depleted PBL, 10⁵ irradiated autologous PBL, and 5×10⁴ irradiated CD4⁺ cloned T cells per well of a 96 well U-bottom plate in the same medium. Six days later 30U/ml recombinant IL-2 was added. Clones were screened for cytotoxicity to the inducing clone and either an autologous lymphoblastoid line or an autologous CD4⁺ clone with a different TCR Vβ and subcloned. Lines and clones were maintained by stimulation every 2 weeks with a mixture of irradiated autologous PBL feeders and the inducing clone in medium containing 30U/ml IL-2.

Cytotoxic Assays

Chromium release assay: ⁵¹Cr release assays were carried out as described (Friedman et al., 1981). Briefly, four days after stimulation CD4⁺ target cells were labeled with 200 mCi ⁵¹Cr (New England Nuclear, Boston, Mass.) for 60 minutes and placed at 2×10⁴ per well per well in 96 well U-bottom plates (Nunc) in triplicate. 10⁵ effector cells (5:1 E:T ratio) were added, and incubation was carried out at 37° C. for 14 hr. Supernatant was harvested and counted in an LKB gamma counter (Pharmacia, Gaithersburg, Md.). The percent specific chromium release was calculated as (sample-spontaneous)/(total-spontaneous)×100. In antibody blocking experiments, chromium release assays were carried out as described in the presence and absence of 20 micrograms/ml purified W6/32 antibody. Targets were incubated with antibody 60 minutes before addition of the effector cell in all cases.

Cold target inhibition assay: 10⁵ per well effector cells (anti-JK50t) were added to 2×10⁴ per well ⁵¹Cr labeled targets (JK50t) in the presence of graded numbers of an unlabeled inhibitor T cell line (ranging from 5×10⁴ to 4×10⁵/well). All assays were carried out in triplicate as above. Specific chromium release was determined and the percent inhibition calculated as (Co−Ci)/Co×100, where Co is the cytotoxicity in the absence and Ci the cytotoxicity in the presence of the cold target inhibitor.

Cytofluorographic Analysis

The methods utilized for cytofluorographic analysis have been described previously (Lederman et al., 1992). Briefly, cells were first treated with aggregated human Ig (Enzyme International, Fallbrook, Calif.) to block nonspecific Ig binding. 5×10⁴ cells were incubated with saturating concentrations of the indicated directly coupled monoclonal antibodies for 10 minutes at 4° C. and rinsed in the presence of propidium iodide (25 μg/ml) in order to eliminate dead cells from analysis. Fluorescence intensity was measured on a FACScan cytofluorograph (Becton Dickinson).

Antibodies

Antibodies to human CD3, CD4 and CD8 conjugated to fluorescein isothionate (FITC) were purchased from Becton Dickinson. Antibodies to TCR Vβ2 and TCR Vβ3 conjugated to biotin were purchased from Amac Inc (Westbrook, Me.). Hybridomas producing the monoclonal antibodies W6/32, L243, and OKT3 were purchased from American Tissue Culture Collection (Rockville, Md.). Ascites containing these antibodies was produced and purified as described (Lederman et al., 1992).

Results

Isolation of Human CD8⁺ Cytotoxic T Lymphocytes (CTL) Specific for Autologous CD4⁺ T Cells Expressing TCR's Belonging to Distinct Vβ Families.

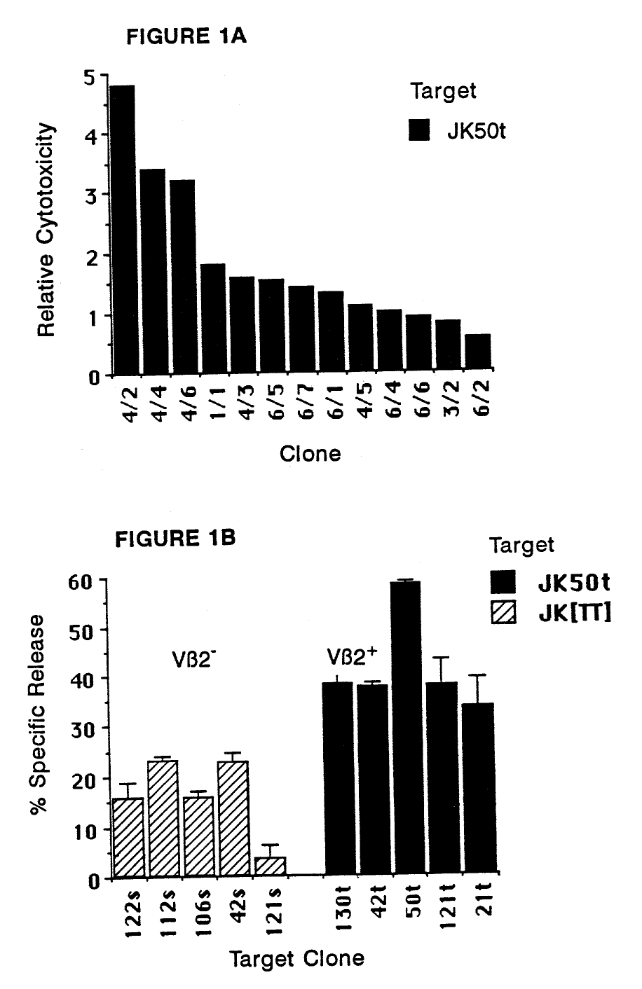

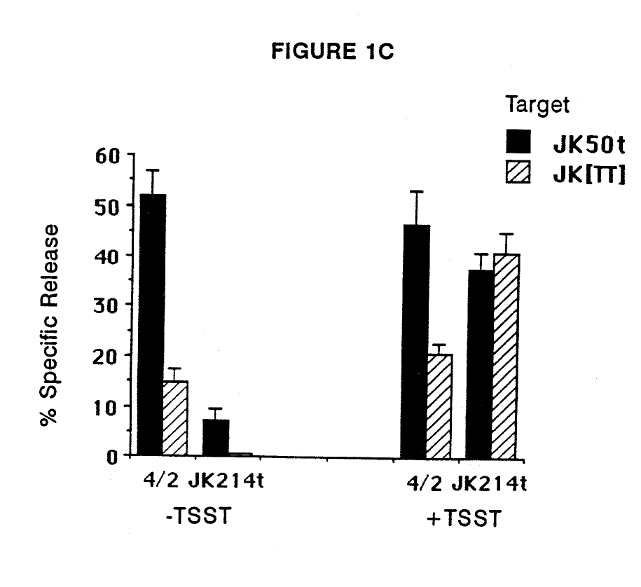

In a first series of experiments, CD4⁺ T cell clones were isolated from a normal donor (JK) by stimulation of peripheral lymphocytes with the superantigens TSST-1 or SEB, which are known to activate T cells expressing mutually exclusive sets of TCR Vβ genes (Vβ2 and Vβ3,12,14,15,17,20 respectively (Marrack and Kappler, 1990)). These were subcloned using their respective superantigens, and representative subclones were subsequently maintained in the absence of feeder cells with periodic stimulation by phorbol/ionomycin. One TSST-1 reactive Vβ2⁺ CD4⁺ clone, JK50t, was used as the inducer for production of the autologous CD8⁺ T cell line, anti-JK50t. This line proliferated in response to JK50t and was initially shown to lyse JK50t but not an autologous lymphoblastoid cell line. Thirteen clones of this CD8⁺ line were obtained by limiting dilution and screened against the inducing JK50t clone as well as the autologous lymphoblastoid line (FIG. 1A). JK4/2, the most specifically reactive against JK50t, was subcloned and tested against a panel of independently isolated autologous Vβ2+ and Vβ2⁻ CD4⁺ T cell clones (FIG. 1B). Reactivity of this subclone was greatest toward the inducing clone JK50t, somewhat less for other Vβ2⁺ CD4⁺ targets, and lowest for Vβ2⁻ CD4⁺ T cells. This result suggests that JK4/2 preferentially recognizes a structure related to TCR Vβ2 which is expressed on autologous CD4⁺ T cell clones.

Although the target cells had been maintained by pharmacological stimulation for many generations, the possibility that the apparent specificity for Vβ2 bearing cells might be due to small amounts of residual superantigen used in their original selection was considered. Therefore the cytotoxicity of JK4/2 was compared toward Vβ2⁺ CD4⁺ and Vβ2 CD4⁺ T cells in the presence and absence of TSST-1 (FIG. 1C). As a positive control we used the CD8⁺Vβ2⁺ T cell clone JK214t, which previously had been shown to kill MHC Class II expressing targets in the presence of TSST-1. Vβ2⁺ CD4⁺ cells were efficiently killed by JK4/2 in the absence of TSST-1, and killing was not increased in its presence. Furthermore, baseline cytotoxicity toward the Vβ2⁻ CD4⁺ cells was not augmented in the presence of TSST-1. On the other hand, JK214t killed all targets in the presence of TSST-1 but not in its absence. Taken together, these experiments rule out the possibility that carryover of TSST-1 from the original isolation of inducer clones contributed significantly to the cytotoxicity of the anti-JK50t subclone JK4/2 to the Vβ2⁺ CD4⁺ cells.

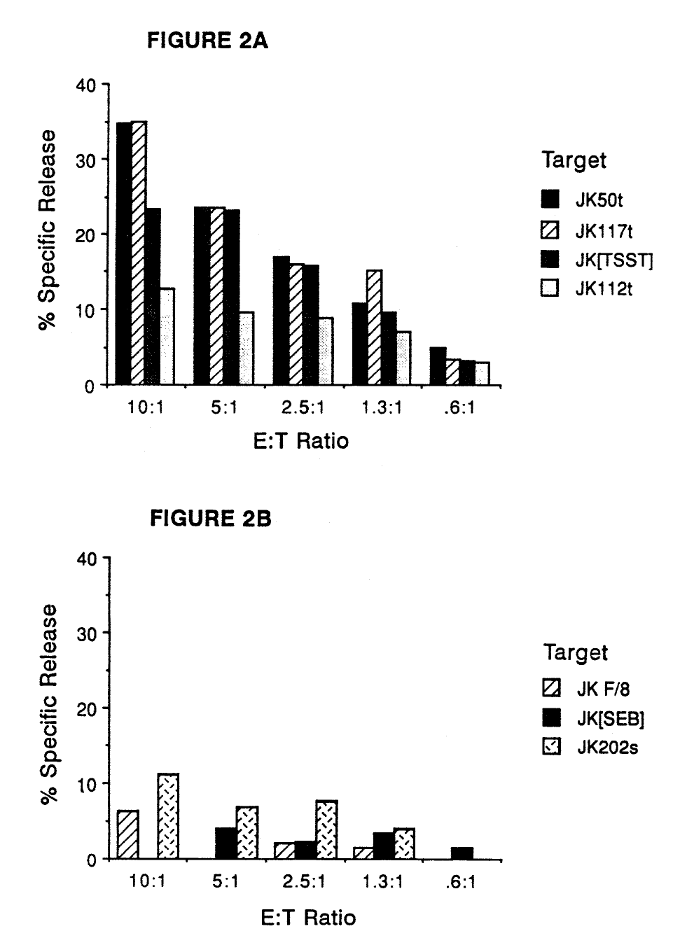

The fact that the anti-JK50t subclone JK4/2 was cytotoxic not only to the inducing JK50t clone but also to other autologous, independently isolated Vβ2⁺ CD4⁺ clones that presumably utilized different Vβ2 clonotypic determinants, but was not cytotoxic to Vβ2⁻ CD4⁺ clones, suggested that JK4/2 does not recognize strictly clonotypic sequences on the targets. To determine if this was a feature unique to JK4/2 or a more general feature of CD8⁺ anti-CD4⁺ T cells, the reactivity of the parent line from which JK4/2 had been cloned was examined. As shown in FIG. 2A, the anti-JK50t parental line was cytotoxic toward cloned Vβ2⁺ CD4⁺ targets (JK50t, JK117t and JK112t) as well as to a polyclonal Vβ2⁺ CD4⁺ T cell line (JK(TSST)), whereas it was minimally active against either cloned Vβ2⁻ CD4⁺ targets (JKF8, JK202s) or to a polyclonal Vβ2⁻ CD4⁺ target (JK(SEB)) (FIG. 2B). The fact that the anti-JK50t line, which was raised against a single Vβ2⁺ CD4⁺ target, was cytotoxic to a polyclonal population of Vβ2⁺ cells is suggestive that sequences shared by Vβ2 T cell receptors are sufficient to identify a cell as a suitable target. Together with the clonal analysis described above, this further substantiates the idea that the target structure recognized by these CD8⁺ T cells is a determinant shared by a large proportion of Vβ2⁺ cells.

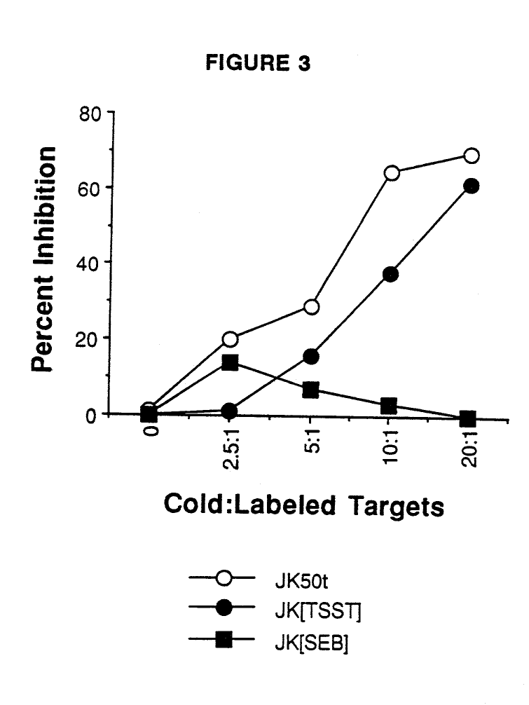

In order to further study the specificity of the anti-JK50t line, cold target inhibition assays were carried out using both cloned and polyclonal CD4⁺ cells as the cold target inhibitors. In these experiments the polyclonal anti-JK50t parental cell line was used as the effector and the inducing CD4⁺ clone JK50t as the labeled target at a ratio of 5:1. Unlabeled potential cold target inhibitors were added in graded numbers. As shown in FIG. 3, the JK50t clone was efficiently killed by the anti-JK50t parental cell line. Furthermore, a polyclonal Vβ2⁺ CD4⁺ population of cells (JK(TSST)) served as an effective cold-target inhibitor. In contrast the polyclonal Vβ2⁻ CD4⁺ line (JK(SEB)) did not significantly block lysis. Together these data further support the notion that the structure recognized on target cells by the anti-JK50t parental cell line and its subclone JK4/2 is related to determinants common to TCR Vβ2 molecules.

The Susceptibility of Target Cells to TCR Vβ-Directed Cytotoxicity is Dependent on Their State of Activation.

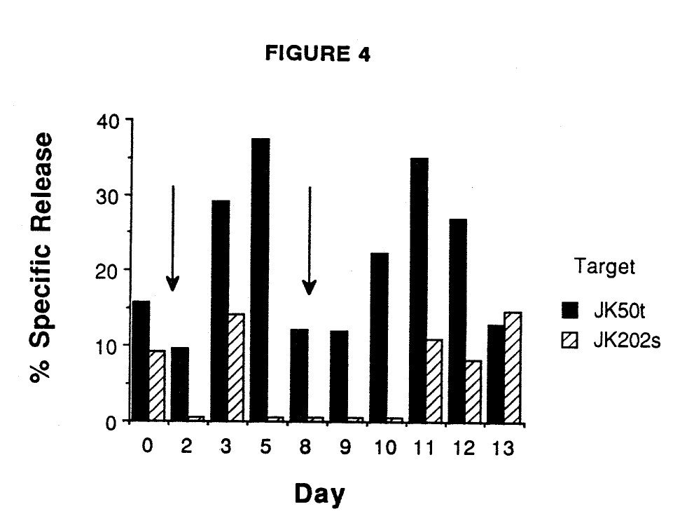

During the course of these experiments it was noted that the sensitivity of Vβ2⁺ CD4⁺ T cells to lysis was related to the time between their restimulation with phorbol/ionomycin and analysis. To study this in greater detail the cytotoxicity of the anti JK50t line was determined towards a Vβ2⁺ CD4⁺ (JK50t) and a Vβ2⁻ CD4⁺ (JK202s) clone over two sequential cycles of CD4⁺ T cell stimulation. As shown in FIG. 4, the TCR Vβ2⁺ target was minimally lysed by the anti-JK50t line for the first two days following activation. Susceptibility was gained from day 3 to day 5 and was lost until two to three days following the subsequent stimulation. On the other hand, the Vβ2⁻ clone was not significantly killed at any time. This result supports the idea that the susceptibility of a CD4⁺ T cell clone to lysis by autologous TCR Vβ specific CD8⁺ cells depends on the state of CD4⁺ T cell activation.

Specific Reactivity of CD8⁺ T Cells Toward Autologous CD4⁺ T Cells is Not Unique to TCR Vβ2-Expressing Targets.

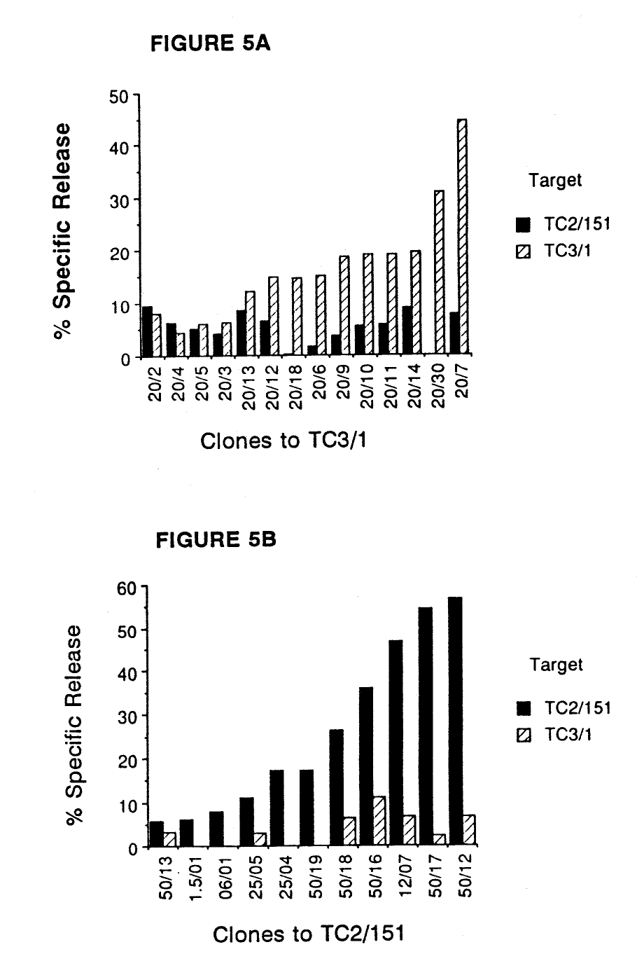

In order to determine whether the phenomenon of CD8⁺ T cell reactivity to autologous CD4⁺ T cells is limited to TCR Vβ2 bearing T cells, both Vβ2⁺ and Vβ3⁺ CD4⁺ clones were first isolated from another donor. These CD4⁺ T cells clones were used to generate CD8⁺ anti-CD4 clones by limiting dilution. CD8⁺ T cells growing in response to the autologous CD4⁺ clones were then screened for cytotoxicity against both the Vβ2+ and Vβ3⁺ CD4⁺ target clones (FIGS. 5A and 5B). As shown, 9 out of 14 CD8⁺ clones raised against the Vβ3⁺ CD4⁺ clone TC3/1 showed greater cytotoxicity to the Vβ3⁺CD4⁺ clone (TC3/1) than to the Vβ2⁺ CD4⁺ clone (TC2/151). The 5 other clones raised to TC3/1 showed less than 10% reactivity toward both targets. Reciprocally, 8 of 11 CD8⁺ clones raised against the Vβ2⁺ CD4⁺ clone showed greater cytotoxicity toward the Vβ2⁺ clone than to the Vβ3⁺ target. Three clones had activity of less than 10%. From these experiments it was conclude that the Vβ specific cytotoxicity of CD8⁺ T cells to autologous CD4⁺ T cells is not limited to CD4⁺ T cells expressing TCR Vβ2.

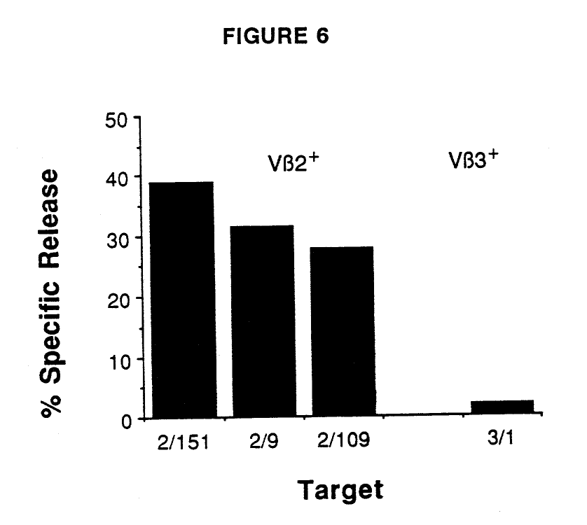

Moreover, additional specificity studies on the anti-Vβ2 clone, TC12/7 were performed. As with the anti-Vβ2 subclone JK4/2 described above, TC12/7 showed maximal cytotoxicity to the inducing Vβ2⁺ CD4⁺ clone, somewhat less to other independently isolated Vβ2⁺ CD4⁺ autologous clones, and little to an autologous clone using a different TCR Vβ (FIG. 6). This result verifies in an independent donor that Vβ specific cytotoxicity of CD8⁺ T cells to autologous CD4⁺ T cells is not strictly anti-idiotypic.

The Interaction Between CD8⁺ CTL and Vβ-Expressing CD4⁺ Cells is Not Blocked by Antibody to Human HLA A/B/C Molecules.

Because CD8⁺ CTL usually recognize antigen in the context of class I MHC molecules, the dependence of the CD8⁺ anti-CD4⁺ CTL described in this study on the recognition of MHC Class I molecules was investigated. The possible MHC Class I involvement in blocking experiments was tested using the monoclonal antibody W6/32, which is known to react with a nonpolymorphic determinant common to human HLA A/B/C molecules. Table 1 gives the result of five independent experiments. The positive control for all five of these experiments consisted in assaying the capacity of W6/32 to block the MHC Class I-dependent cytotoxicity of a CD8⁺ clone (TC9) against an autologous lymphoblastoid line. As shown (Table 1) in all experiments W6/32 markedly blocked killing by the control clone TC9. As a negative isotype control, the monoclonal antibody L243 which reacts with HLA class II molecules had no effect. Although W6/32 effectively blocked the killing mediated by the MHC dependent CD8⁺ clone, it had no significant effect on the cytotoxicity of the Vβ2 specific CD8⁺ CTL clone (TC12/7) to its inducing Vβ2⁺ CD4⁺ T cell target (Table 1). Inhibition was shown not to be masked by a concomitant antibody dependent cellular cytotoxic reaction mediated by MAb W6/32 because the antibody did not induce lysis of the Vβ3⁺ CD4⁺ clone (TC3/1, Experiment 3). Taken together, these experiments show that the Vβspecific CD8⁺ anti-CD4⁺ CTL described here do not interact with the human HLA class I molecules recognized by W6/32. These data are consistent with the results in Example 2 showing that cytotoxicity mediated by murine Vβ specific CD8⁺ anti-CD4⁺ CTL are not blocked by antibody recognizing conventional H-2 haplotypes.

TABLE 1
Failure of MAb W6/32 to Block Cytotoxicity of
TC12/7 to its Inducing Clone*.

| | Effector: | | % | |
|---|---|---|---|---|
| Exp | Target | % | Cytotoxicity | % |
| # | Pair | Cytotoxicity | +W6/32 | Inhibition |
| 1 | 12/7:2/151 | 28.2 | 34.6 | <1 |
| | Control^{¶} | 61.0 | 19.6 | 68 |
| 2 | 12/7:2/151 | 45.9 | 53.6 | <1 |
| | Control^{¶} | 51.9 | 1.2 | 98 |
| 3 | 12/7:2/151 | 23.4 | 26.8 | <1 |
| | 12/7:3/1^{§} | 3.1 | 1.5 | — |
| | Control^{¶} | 56.8 | 17.6 | 68 |
| 4 | 12/7:2/151 | 16.9 | 15.5 | 12 |
| | Control^{¶} | 65.1 | 4.8 | 92 |
| 5 | 12/7:2/151 | 37.6 | 35.0 | 7 |
| | Control¶ | 64.5 | 23.4 | 64 |
| *The 2 CD8⁺ clones TC 12/7 and TC9 were tested against their specific targets, TC2/151 and the lymphoblastoid line TC(LCL) respectively, in a 14 hour chromium release | | | | |
| # cytotoxic assay in the presence and absence of 20 μg/ml MAb W6/32 (in preliminary experiments, 5 μg/ml was sufficient to maximally inhibit the control cytotoxicity). Five | | | | |
| # independent experiments are shown. | | | | |
| ^{§}In addition, in experiment 3, the target TC 3/1 was included to rule out antibody dependent cytotoxicity | | | | |
| ^{¶}The positive control for all five of these experiments consisted in assaying the capacity of W6/32 to block the cytotoxicity mediated by MHC Class I-dependent cytotoxicity | | | | |
| # of a CD8⁺clone (TC9) against autologous lymphoblastoid cells. As a negative isotype control, the monoclonal antibody L243 which reacts with HLA class II molecules had no effect (data not shown). | | | | |

In Example 1 evidence is provided that TCR Vβ structures common to Vβ families expressed on the surfaces of CD4⁺ T cells are involved in cytolytic interactions of human CD8⁺ T cells with autologous CD4⁺ T target cells. CD8⁺ T cells were expanded using autologous clones and were found specifically to lyse the inducing CD4⁺ T cell clone as well as independently isolated autologous CD4⁺ T cell clones and lines expressing the same but not a different TCR Vβ family. Of interest, CD4⁺ T cell targets were susceptible to lysis for only a limited period of time following activation. Moreover, this Vβ specific cytotoxicity was not blocked by the monoclonal antibody W6/32, which reacts with non polymorphic determinants present on HLA A/B/C Class Ia molecules. Taken together, these results demonstrate the existence of CD8⁺ T cells and/or precursors in human peripheral blood that interact with CD4⁺ T cells based on CD4⁺ TCR Vβ usage.

The Vβ specificity of the CD8⁺ CTL was documented in a number of ways. CD8⁺ T cells raised to Vβ2⁺ CD4⁺ T cell clones were shown to be cytotoxic in chromium release assays to autologous, independently isolated Vβ2⁺ CD4⁺ clones but not to autologous Vβ2⁻ CD4⁺ clones (FIGS. 1A, 1B, 1C, and 6). Specificity was further demonstrated in experiments showing that a CD8⁺ line raised against a Vβ2⁺ CD4⁺ clone was cytotoxic to other autologous Vβ2⁺ CD4⁺ lines and clones but not to Vβ2⁻ CD4⁺ targets (FIGS. 2A and 2B). In addition, a polyclonal Vβ2⁺ line of CD4⁺ cells was shown to be an effective cold-target inhibitor of cytotoxicity of this line to the Vβ2⁺ CD4⁺ clone used in its expansion, whereas a Vβ2⁻ line was not (FIG. 3). Finally, it was demonstrated in a reciprocal cloning experiment that CD8⁺ T cells raised against autologous CD4⁺ T cell clones with different TCR Vβ usage were cytotoxic to their inducing clone but not the reciprocal inducer. Taken together, these experiments indicate that CD8⁺ T cells can kill autologous CD4⁺ T cells based, at least in part, on the recognition of their Vβ sequences.

The precise composition of the target structure recognized by the CD8⁺ cells described here is of considerable interest. Because they are CD8⁺ T cells, the possibility was considered that they would interact with their target cells in the same manner as other antigen-specific CD8⁺ T cells by recognizing peptide bound to a MHC Class I molecule. The previous observations that human CD8⁺ T cell clones can inhibit fresh autologous alloreactive CD4⁺ cells in a Class I MHC-restricted manner is consistent with this notion (Koide and Engleman, 1990). In contrast, it was found that the anti-MHC Class I monoclonal antibody W6/32 does not block the interaction of the Vβ specific CD8⁺ clones with their autologous CD4⁺ T cell targets (FIG. 6). Because W6/32 reacts with shared conformational determinants on the major Class I molecules HLA A/B/C the present results strongly suggest that conventional MHC Class I structures were not involved in the recognition by these CD8⁺ T cell clones. This lack of inhibition by an anti-MHC Class I antibody is similar to two other reports concerning the interaction of CD8⁺ T cells with syngeneic CD4⁺ T cells which also suggest that Class I is not involved. Sun et. al. (Sun et al., 1988) described a lack of inhibition by anti-Class I antibody of a CD8⁺ cell line specifically cytolytic to a MBP-responsive CD4⁺ line in Lewis rats. Also in rats, Kimura and Wilson (Kimura and Wilson, 1984) have described CD8⁺ anti-allo-reactive T cells which were not restricted by the Class I type of the allo-reactive cells. Taken together these studies suggest that other histocompatibility molecules might also be involved in the presentation of T cell receptor peptides (Bloom et al., 1992a; Shinohara et al., 1988). In support of this, EXAMPLE 2 shows in a murine system analogous to the human system described here, that an anti-MHC Class I-a antibody also fails to block cytotoxicity of a Vβ8 specific CD8⁺ T cell line to its Vβ8⁺, CD4⁺ targets, whereas an antiserum directed toward molecules of the Class I-b histocompatibility locus, Qa-1, is an efficient inhibitor. The combined results from both species suggest that CD8⁺ T cells can be isolated which recognize all or part of a TCR VP chain on the surface of CD4⁺ cells independently of classical Class I-a.

The finding that CD4⁺ cells are not continuously subject to cytotoxicity by CD8⁺ cells raised against them but can be efficiently killed for only a limited period of time following their activation (FIG. 4) is significant in understanding the potential relevance of this interaction in vivo. If present in vivo, the restricted period of sensitivity would serve to limit regulation to recently activated CD4⁺ T cells. Thus naive as well as memory T cells would not be subject to the T-T cell interactions described here. One possible explanation for this finding is that the T cell receptor target structure itself is not continuously present on the CD4⁺ T cell surface. For example, the expression of Class I molecules on lymphoid cells is not constant. The surface density of Class I molecules on human CD4⁺ T cell clones is transiently increased following activation, as is susceptibility to lysis. In addition, certain MHC Class I-b molecules in both humans and in mice are present only on activated, but not resting, lymphocytes (Paul et al., 1987, Shawar et al., 1994). Class I molecule expression is also influenced by cytokines. Beta-interferon, which has been shown to be effective in the treatment of multiple sclerosis (The IFNB Multiple Sclerosis Study Group, 1993), rapidly induces an increase in Class I molecules on human CD4⁺ T cells (Follows et al., 1979) and in Class I (Lindahl et al., 1974) and in Qa-1 molecules (Stanton and Carbon, 1983) on murine T cells. If MHC molecules of this type were utilized in the presentation of a TCR-related target structure, recognition would depend on the state of activation of the CD4⁺ T cell. On the other hand, the temporal sensitivity of CD4⁺ cells may simply reflect an activation-dependent appearance of non-specific molecules required for adequate conjugate formation or for apoptosis. In either case, our results show that CD4⁺ cells are not targets for indiscriminate attack by Vβ specific CD8⁺ cells simply on the basis of their T cell receptor variable chain usage.

Of equal importance to the overall target structure is the region of the CD4⁺ TCR Vβ molecule which is involved. Early models of immune regulation included often complex circuitry involving interactions among T cells and soluble T cell factors based on idiotypic anti-idiotypic recognition (Dorf and Benacerraf, 1984). Some experiments in humans have indicated that interactions among autologous T cells may occur on a purely clonotypic basis (Naor et al., 1991). In contrast, recent experiments in which animals were vaccinated with peptides representing portions of the Vβ determinants of a TCR molecule indicate that Vβ sequences can induce immunoregulatory events including down-regulation of the CD4⁺ cells expressing the corresponding TCR Vβ (Vandenbark et al, 1989; Howell et al., 1989; Gaur et al.,1992; Gaur et al.,1993). The results presented here, in which potentially regulatory CD8⁺ T cells are expanded in vitro using autologous T cell clones as the only stimulus, indicate that CD8⁺ T cells responsive to CD4⁺ cells purely on the basis of the CD4⁺ Vβ chain exist. However the cytotoxicity of these cells was greatest toward the inducing clone, but somewhat less to independently isolated clones and lines expressing the same TCR Vβ (FIGS. 1A, 1B, 1C, 2A, 2B, 5A, 5B, 6). In the case of the polyclonal CD8⁺ effector cells, this specificity reflects the presence of distinct clones directed toward either clonotypic or framework specificities of the inducer cell. However, the observation that in two subcloned effector clones this same preference for the inducer clones was observed suggests the possibility that the TCR Vβ sequences recognized may involve both clonotypic as well as non-clonotypic regions. In this regard it is of interest that in the murine system it has recently been shown that following immunization with myelin basic protein, CD4⁺ T cells arise which proliferate in response to peptides representing framework sequences of the TCR Vβ8.2 molecule, but only to those sequences which are near the junctional region (Kumar and Sercarz, 1993). Additional fine-structural analysis will be required to define the precise contribution of different regions of TCR variable regions to the T-T cell interactions involved in the murine studies as well as in the human.

The in vivo significance of the CD8⁺ T cells described here is of great interest particularly since there have been a number of reports in animal systems regarding the ability of CD8⁺ cells to regulate the immune response of CD4⁺ cells on the basis of their TCR. In rats and in mice, EAE is directly caused by myelin basic protein-responsive CD4⁺ cells utilizing primarily the Vβ8.2 T cell receptor (Kumar and Sercarz, 1993; Sun et al., 1988). Mice which recover from this disease are protected both from relapses and from a second induction by immunization with MBP, but only if they possess CD8⁺ cells (Jiang et al., 1992; Koh et al., 1992). Similarly, vaccination of mice with a peptide representing the CDR2 region of the TCR Vβ8.2 molecule leads to functional inactivation of all Vβ8.2+CD4⁺ cells, but not Vβ8.2⁻ CD4⁺ cells, only in the presence of a normal population of CD8⁺ cells (Gaur et al., 1993). Moreover, it has been shown that clones of CD4⁺ cells with reactivity to certain peptides of the TCR Vβ8.2 molecule can adoptively transfer resistance to induction of EAE in mice only if the recipient animals have CD8⁺ cells (Kumar and Sercarz, 1993). Finally, the results from EXAMPLE 2 demonstrate that the prolonged depletion of peripheral Vβ8⁺ CD4⁺ T cells that follows the administration of SEB is inhibited by in vivo treatment with anti-CD8 monoclonal antibody. Thus, in several different mammalian systems it has been shown that immune regulation by the classical suppressor/cytotoxic subset of CD8⁺ cells is directed towards CD4⁺ cells that share TCR variable chain sequences. The results presented here and in EXAMPLE 2 showing direct interaction between CD8⁺ and CD4⁺ cells based on the TCR Vβ usage of the CD4⁺ cells provides a mechanism for these observations.

### EXAMPLE 2

To more readily study the role of CD8⁺ T cells in regulating CD4⁺ T cells, the involvement of CD8⁺ T cells in the deletion of CD4⁺ T cells expressing the Vβ8 TCR in SEB treated animals was investigated. Two kinds of CD8⁺ T cell deficient animals were used to study the role of CD8⁺ T cells in regulating CD4⁺ T cells in vivo. First, in experiments analogous to those described in EAE, mice depleted of CD8⁺ T cells by injection of anti-CD8 antibody in vivo (Jiang et al., 1992) were investigated. Second, β₂m-/- mice which are known to be deficient in CD8⁺ T cells (Zijlstra et al., 1990) were studied. In both systems evidence was provided that CD8⁺ T cells participated in SEB induced deletion of CD4⁺Vβ8⁺ T cells in vivo. In an attempt to understand the possible mechanisms involved in such T-T cell interactions, an in vitro cytolytic system was established to investigate whether CD8⁺ T cells stimulated by SEB-activated autologous CD4⁺Vβ8⁺ T cells will specifically kill CD4⁺ T cell targets expressing Vβ8 TCR. It was demonstrated that CD8⁺ T cells derived from SEB primed mice could be restimulated in vitro and were cytotoxic to the specific CD4⁺ T cell targets based on their TCR Vβ expression. Furthermore, this autologous TCR Vβ specific cytolysis was B₂-microglobulin dependent. Quite surprisingly, this cytolytic interaction between CD8⁺ effector cells and CD4⁺ targets was blocked by antisera to a MHC Class I-b molecule, Qa-1, but not by antibody to classical MHC class I-a molecules. Thus, these data indicate that a subset of CD8⁺ T cells can be induced by activated CD4⁺ T cells to kill the inducer CD4⁺ T cells through the recognition of their TCR Vβ chains or the peptides derived from their TCR Vβ chains in conjunction with Qa-1.

Experimental Procedures

Animals

BALB/c mice, C57BL/6J (B6) mice, β₂m-/- mice and 129B6F2/J control mice, (female, 6-12 wk old), were purchased from Jackson Lab, and maintained in our animal facilities.

Antibodies and Antisera

Fluorescein (Fl) or Allophycocyanin (APC) 53-6.72 (anti mouse CD8), Fl-34.1.2 (anti mouse H-2d), APC-GK1.5 (anti mouse CD4), biotin(bio)-F23.1 (anti mouse TCR Vβ8.1-3), Bio-KJ-16 (anti mouse TCR Vβ8.1,2), and Bio-RR3.15 (anti mouse Vβ11) were purified from the ascites of correspondent hybridomas and conjugated in our laboratory. Bio-RR4.7 (anti mouse TCR Vβ6) was purchased from Pharmingen (San Diego, Calif.). M1/42.39 was purified from the supernatant of the hybridoma culture using a protein G column. Anti-Qa-1a and anti-Qa-1b antisera were prepared as described previously (Boyse et al., 1972; Stanton and Boyse, 1979; Eardley et al., 1978).

Cell Lines

L3 cells, an allo-reactive CD8⁺ CTL line, from C57BL/6J strain, specific to H-2d alloantigen, were kindly provided by Dr. Gerald Siu of Columbia University (Glasebrook and Fitch, 1979). These cells were cultured in Clicks' HEAA medium (Irvine Scientific, Santa Ana, Calif.), supplemented with 10% FCS, 100 μl/ml of penicillin and streptomycin, 2 mM b-mercaptoethanol and 25 μl/ml of human IL-2 (D&R Systems Inc. Minneapolis, Minn.). The L3 line was maintained by stimulation with irradiated (3,000 Rads) spleen cells from BALB/c mice at a 1:5 to 1:10 ratio, every 10 to 14 days. The P815 (H-2d) and EL4 (H-₂b) murine cell lines were obtained from the ATCC, maintained in RPMI 1640 medium supplemented with 10% of FCS, 100 μl/ml of P/S and 2 mM b-mercaptoethanol.

Generation of TCR Vβ8 Specific CTL Lines

Pooled spleen and lymph node cells from 4 SEB-primed mice (4-14 days after SEB injection) were first depleted of B cells, by incubating with goat anti-mouse immunoglobulin (Ig) and goat anti-rat Ig coated magnetic beads (Advanced Magnetic Inc. Cambridge, Mass.) at a ratio of 1×10⁷ cells per ml of beads for 30 min at 4° C. The cells were separated from beads by a Bio Mag Separator (Advanced Magnetic Inc. Cambridge, Mass.), and the unbound cells were then depleted of Vβ8⁺ T cells by using the anti-mouse TCR Vβ8 Mab, F23.1(1×10⁷/10 g). The cells from the second round of negative selection were positively selected for CD8⁺ cells by incubating 1×10⁷ cells with 0.1 ml of 1:40 ascites of 53-6.72 (anti mouse CD8) followed by incubation with goat anti rat coated beads and subsequent magnetic separation. The cells bound to the beads were incubated overnight at 370C to allow their dissociation from beads. The final purified population consisted of CD8⁺V18⁻ T cells (>95%). The CD8⁺Vβ8⁻ T cells were then incubated with irradiated (3,000R) SEB-activated syngeneic CD4⁺Vβ8⁺ T cell line (5-10 days after SEB stimulation) and APCs (syngeneic spleen cells) in a 1:1:1 ratio, at 37° C., 6% CO2 for 14 days. IL-2 (10 U/ml) (D&R Systems Inc. Minneapolis, Minn.) was added on day 3 of the culture.

Generation of SEB and SEE-Induced CD4⁺ target T Cell Lines

Spleen and lymph node cells from naive mice were first depleted of CD8⁺ T cells and then positively selected for TCR Vβ8⁺ T cells, or negatively selected for TCR Vβ8⁻ T cells. CD4⁺Vβ8⁺ T cells were stimulated with SEB (lg/ml) (Toxin Technology, Madison, Wis.); CD4⁺Vβ8⁻ T cells were stimulated with SEE (0.1 μg/ml)(Toxin Technology); and CD4⁺ T cells were stimulated with both SEB (1 μg/ml) and SEE (0.025 or 0.1 μg/ml) or SEB (1 μg/ml) alone. In all SEB or SEE primed T cell cultures, irradiated (3,000R) spleen cells were added as APCs. After 3 days, T cell blasts were isolated and cultured in complete IMDM medium supplemented with IL-2 (10 U/ml). The phenotypes of these T cells were determined by staining the cells with APC-GK1.5, Fl-53-6.72 and Bio-F23.1 or Bio-RR-3.15 (anti-Vβ11) and analyzed by FACS.

FACS Analysis for Detecting SEB-Induced TCR Vβ Specific T Cell Death in Peripheral Blood

Mice were injected in the tail vein on day 1 with either 50 μg SEE in 0.1 ml PBS or PBS alone. Seven days before SEB/PBS injections one group of mice were CD8⁺ T cell depleted as previously described (Jiang et al., 1992). On days 4,7,14 and 21 after priming the mice were examined for numbers of CD4⁺Vβ8.1,2⁺; CD8⁺Vβ8.1,2⁺, CD4⁺Vβ6⁺ and CD8⁺Vβ6⁺ T cells. At each time point peripheral blood drawn from the tail vein of 4-6 mice in each group was pooled, mononuclear cells purified on a lymphocyte separation medium gradient (Organon Technicka, Durham, N.C.) and stained with the following monoclonal antibodies: fluorescein (Fl) 53-6.72 (anti-CD8); Allophycocyanin (APC) GK1.5 (anti-CD4); and biotin (Bio) KJ-16 (anti-Vβ8.1,2) or biotin RR4.7 (anti-Vβ6). Biotin conjugates were revealed with Texas Red-Avidin. Dead cells were excluded using propidium iodide. Flow cytometric analysis was performed on a dual laser FACStar plus (Becton-Dickinson, San Jose, Calif.) using FACS/DESK data analysis software (Stanford University). The data are expressed as the percent of total CD4⁺ or total CD8⁺ T cells.

FACS Analysis for Measuring Specific CTL Activity

To assay the L3 cell line for H-2d allospecific killing we used the H-2d expressing target, P815, a CD8⁻ mastocytoma cell line derived from DBA/2 mice as the positive target. El-4, a H-2b expressing CD8⁻ T lymphoma line served as the negative target. Graded numbers of L3 effector cells were added to a fixed ratio of H-2^{d+} (specific targets) to H-2^{d-} (non-specific targets) for 4 hours prior to FACS analysis (see below). The reduction in the ratio of H-2^{d+}/H-2^{d-} cells in the presence of effector cells, compared to the ratio of target cells in the absence of effectors, served as a measure of cell death.

In the TCR Vβ8 specific cytolytic system, SEB activated CD4⁺Vβ8⁺ T cells were used as specific Vβ8⁺ targets. In addition, SEB plus SEE or SEB alone were used to activate CD4⁺Vβ8⁻ T cells. These CD4⁺V8⁻ served as non-specific Vβ8⁻ targets. In this system the target T cells used were from day 4-6 superantigen activated (SEB or SEE) cultures because we had noted that susceptibility to lysis varied as a function of time following superantigen activation and peaked between days 4 and 6. Graded numbers of putative CD8⁺ CTL effector populations were then added to Vβ8⁺ and Vβ8⁻ targets for 24 hours and the ratio of Vβ8⁺/Vβ8⁻ cells was measured by FACS. The baseline control was the ratio of Vβ8⁺/Vβ8⁻ after 24 hours in the absence of effector cells.

In both CTL systems, triplicate or duplicate samples were set up for each E/T ratio. Following effector to target cell incubation two color fluorescence was used to distinguish targets from effectors as well as specific from nonspecific targets. Targets and effectors were distinguished by expression of CD8 using the conjugated anti-CD8 antibodies Fl-53-6.72 or APC-53-6.72 antibodies. In the L3 system, cells were stained with F-34.1.2 (anti H-2d) to distinguish P815 (H-2d) from EL4 (H-2b). In TCR Vβ specific cytolytic system, cells were stained with Bio-F23.1 to distinguish Vβ8⁺ targets from Vβ8⁻ targets, or Bio-RR3.15 to distinguish Vβ11⁺ targets from Vβ11⁻ targets. Biotin conjugated reagents were revealed with Texas Red-Avidin. Dead cells were excluded using propidium iodide. For data analysis, the CD8⁺ T cells were gated out and the data was expressed as the ratio of the positive stained cells (specific targets) versus negative stained cells (non-specific targets). The percentage of the specific cytolysis of specific targets was calculated as:{[(positive stained cells/negative stained cells of control group)−(positive stained cells/negative stained cells of experimental group)]/(positive stained cells/negative stained cells of control group)}×100%.

⁵¹Cr Release Assay

P815, EL4, SEB activated CD4⁺Vβ8⁺ T cells, and SEE activated CD4⁺Vβ8 T cells were labeled with 200 μCi ⁵¹Cr (New England Nuclear, Boston, Mass.) for 45-60 minutes, washed three times and used as targets. L3 effector cells and TCR Vβ8 specific CD8⁺ CTL cells were added to corresponding targets at varying E/T ratio and incubated at 37° C. for either 4 hrs (standard assay) or 12 hrs (special assay). In lectin induced MHC class I independent cytolysis, ConA was added at the beginning of the culture (20-40%g/well). After incubation, the supernatants were harvested, and radioactivity was counted on a gamma counter (LKB, Piscataway, N.J.). The mean of triplicate samples was calculated and the percent specific ⁵¹Cr-release was determined as follows: % of specific cytolysis=[(experimental ⁵¹Cr-release−control ⁵¹Cr-release)/(maximum ⁵¹Cr-release−control ⁵¹Cr-release)]×100%. Experimental ⁵¹Cr release represents counts from target cells mixed with effector cells, control ⁵¹Cr-release represents counts from targets incubated with medium alone (spontaneous release), and maximum ⁵¹Cr-release represents counts from targets exposed to 5% Triton X-100.

Results

The in vivo Delayed Deletion of CD4⁺Vβ8⁺ T Cells Following SEB Administration is Markedly Reduced by in vivo Treatment of Mice with Anti-CD8 Antibody.

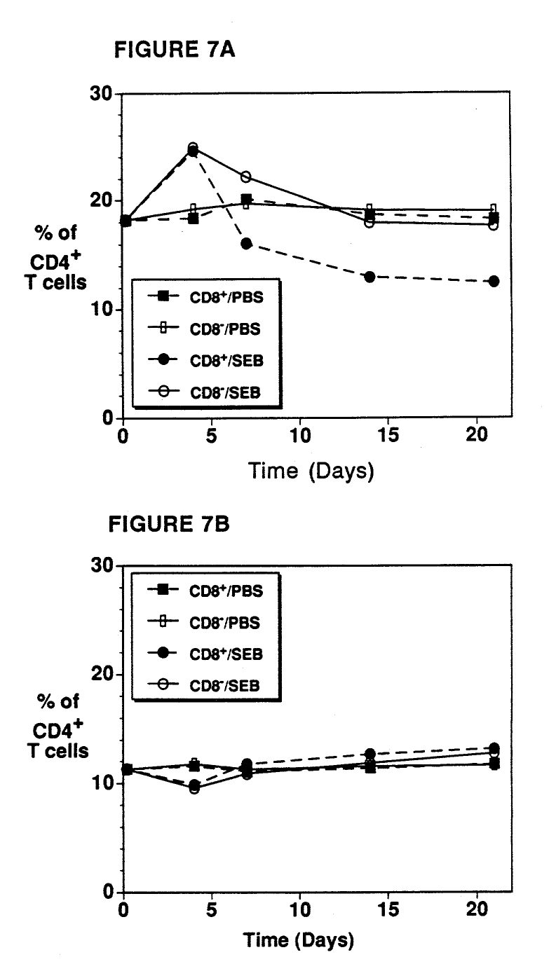

Following treatment of animals with SEB there is an initial rapid deletion of CD4⁺ T cells followed by an expansion of those T cells that express the characteristic Vβ segment reactive with SEB. Subsequently, there is a delayed deletion below background level of the T cells expressing the Vβs reactive with SEB (Gonzalo et al., 1994; Kawabe and Ochi, 1991; Rellahan et al., 1990). To address whether CD8⁺ T cells are involved in this delayed TCR Vβ restricted deletion following SEB administration to mice, the effect of SEB on CD4⁺Vβ8⁺ populations of T cells in normal mice and in mice depleted of CD8⁺ cells were compared by in vivo administration of anti-CD8 antibody. 50 μg SEB was injected intravenously into both untreated and CD8⁺ T cell depleted mice and the portion of CD4⁺Vβ8.1,2⁺ T cells (which are specifically interactive with SEB) in peripheral CD4⁺ T cells of those mice was determined. As shown by FIG. 7A, and Table 2, in SEB primed CD8⁺ mice, the number of CD4⁺Vβ8.1,2⁺ T cells increased until day 4 and then decreased reaching a low point between day 7 and day 14, and remained low for at least another week. In the CD8⁺ T cell depleted mice treated with SEB, the number of CD4⁺Vβ8.1,2⁺ T cells also increased after SEB injection, reached a peak on day 4, and then slowly returned to baseline levels by day 14. The decrease (25-30%) below normal levels in the percentage of CD4⁺Vβ8.1,2⁺ T cells that is observed in normal mice treated with SEB, was completely eliminated by the depletion of CD8⁺ T cells. Both SEB induced T cell death and the protection due to CD8⁺ T cell depletion were TCR Vβ-specific, because there was no difference in the numbers of CD4⁺Vβ6⁺ T cells between CD8⁺ T cell non-depleted and CD8⁺ T cell depleted, SEB primed mice (FIG. 7B and Table 2). These data confirm that following SEB injection in normal mice there is an initial proliferation of CD4⁺Vβ8⁺ T cells followed by a decrease in the number of CD4⁺Vβ8⁺ cells below baseline. Further, the data demonstrate that the decrease of CD4⁺Vβ8⁺ T cells below baseline is CD8⁺ T cell dependent because this decrease is observed only in untreated mice containing CD8⁺ T cells, but not in CD8⁺ T cell depleted mice.

TABLE 2
Analysis of Vβ8.1,2 and Vβ6 Expression on Peripheral T Cells
in CD8^{+T Cell-Nondepleted and CD8}^{+T Cell-Depleted}
BALB/c MicePHU a

| | | Percentage of Total | | | |
|---|---|---|---|---|---|
| | | CD4⁺ T Cells | | CD8⁺ T Cells | |
| Anti- | | | | | |
| CD8 | | | | | |
| Treat- | Injec- | | | | |
| ment | tion | Vβ8.1,2 | Vβ6 | Vβ8.1,2 | Vβ6 |
| No | PBS | 18.7 ± 0.84 | 11.4 ± 0.20 | 25.5 ± 0.49 | 12.2 ± 1.4 |
| Yes | PBS | 19.1 ± 0.21 | 11.6 ± 0.32 | — | |
| No | SEB | 13.0 ± 2.40 | 12.7 ± 0.44 | 29.1 ± 4.8 | 11.6 ± 3.0 |
| Yes | SEB | 18.0 ± 0.31 | 11.9 ± 0.90 | — | — |

^{a}Experiments were done as in FIGS. 7A and 7B. This table shows the data of expression of Vβ8.1,2 and Vβ6 in CD8^{+ T cell-nondepleted and CD8}^{+ T cell-depleted mice 14 days after SEB injection. Each value represents data of 12-18 mice from three separate experiments.}

TABLE 3
Analysis of Vβ8.1,2 TCR Expression on Peripheral CD4⁺ T
Cells in μ₂m^{-/-} mice after SEB Stimulation^{a}

| Experiment | | | CD4⁺ Vβ8⁺/ Total CD4⁺ | | | | | |
|---|---|---|---|---|---|---|---|---|
| num- | | | (Percentage) Time (Days) | | | | | |
| ber | Mice | Injections | 4 | 7 | 14 | 21 | 60 | 120 |
| 1 | | | | | | | | |
| | B6 | PBS | 18.6 | 18.8 | 18.6 | 18.5 | — | — |
| | B6 | SEB | 18.1 | 11.2 | 12.2 | 13.0 | — | — |
| | β₂m^{-/-} | PBS | 18.5 | 18.6 | 18.6 | 18.5 | — | 19.1 |
| | β₂m^{-/-} | SEB | 24.2 | 18.7 | 18.5 | 18.6 | — | 19.3 |
| 2 | | | | | | | | |
| | (BE × 129)F2 | PBS | 18.6 | 18.7 | 19.0 | 18.9 | 18.8 | — |
| | (BE × 129)F2 | SEB | 17.0 | 12.5 | 13.3 | 13.5 | 14.3 | — |
| | B₂m^{-/-} | PBS | 18.4 | 18.9 | 18.6 | 18.8 | 19.1 | — |
| B₂m^{-/-} | SEB | 20.2 | 20.0 | 19.1 | 18.9 | 18.4 | — | |

^{a}Experiments were done as described in FIGS. 7A and 7B. B6 mice and (B6 × 129)F2 mice served as controls. There were 4 mice per group in each experiment.

The in vivo Deletion of CD4⁺Vβ8⁺ T Cells Following SEB Administration is Abrogated in β₂m-/- Mice.

To further verify the importance of CD8⁺ cells in the in vivo deletion below baseline of CD4⁺V8⁺ cells following SEB administration, β₂m-/- mice known to lack mature CD8⁺ T cells were studied. The β₂m-/- mice used were derived from a C57BL (B6) X 129 cross and are H-2b (Zijlstra et al., 1989; Muller and Koller, 1992). Both B6 and (B6×129)F2 mice were used as control animals. In two separate experiments peripheral CD4⁺Vβ8.1,2⁺ cells in SEB primed β₂m-/- mice increase on day 4, return to and remain at normal level after day 7 (Table 3). In contrast the CD4⁺Vβ8.1,2⁺ cells are reduced to greater than 30% of baseline in both SEB primed control B6 and (B6 X 129)F2 mice. It is curious that in these experiments, unlike previous experience in BALB/c mice, there was no CD4⁺Vβ8⁺ T cell expansion observed by day 4 in both B6 and (B6 X 129)F2 mice. It is possible that Vβ8 specific CD8⁺ T cells involved in the regulation of CD4⁺ T cells arise earlier in these mice.

The in vitro Generation of CD8⁺ Killer Cells Specific for CD4⁺Vβ8⁺ Target Cells.

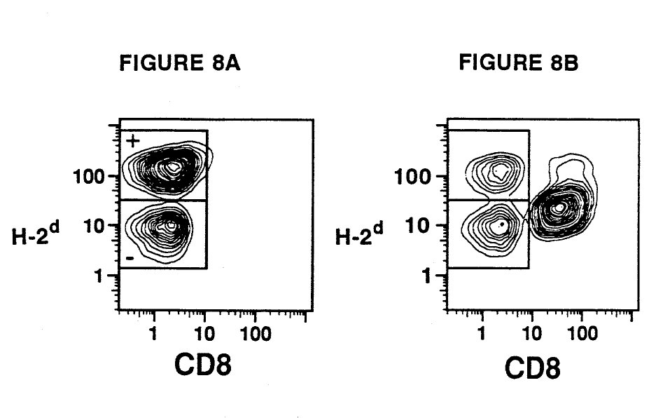

The requirement for CD8⁺ T cells made it unlikely that the deletion of CD4⁺Vβ8⁺ T cells following SEB administration was only due to an endogenous self-destruction program activated by SEB. Moreover, the specificity of the deletion raised the possibility that the initial SEB induced CD4⁺Vβ8⁺ T cells further stimulated a population of CD8⁺ T effector cells with anti-Vβ8 specificity (distinct from the CD8⁺Vβ8⁺ T cells directly stimulated by SEB). It was hypothesized that these CD8⁺ T cells could then mediate the deletion of CD4⁺Vβ8⁺ T cells by a cytotoxic mechanism. To test this hypothesis in vitro, a CD4⁺Vβ8⁺ T cell line was established by stimulating purified CD4⁺Vβ8⁺ T cells with SEB. This line was then irradiated and used to induce CD8⁺Vβ8⁻ T cell lines in vitro. Since the superantigen SEE does not react with T cells bearing Vβ8 TCR (Marrack and Kappler, 1990), SEB and SEE were used to obtain CD4⁺ T cell lines bearing different TCR Vβ specificities including the generation of Vβ8⁺ and Vβ8⁻ cell lines. To address the function and specificity of these CD8⁺ T cell lines their ability to specifically kill the CD4⁺ stimulating T cells that were used to induce them in the first instance was investigated. In preliminary studies the CD8⁺ T cells initially induced by CD4⁺Vβ8⁺ cells could specifically kill CD4⁺Vβ8⁺ targets to a greater extent than CD4⁺Vβ8⁻ targets in a conventional ⁵¹Cr release assay but only after prolonged periods of incubation (>12 hours). However, incubation time greater than 12 hours were often associated with a high spontaneous release of ⁵¹Cr from targets which significantly reduced the signal-to-noise level, increased the experimental variability and made the interpretation of experiments difficult. During the course of these studies it was observed that the reduction (due to lysis) of CD4⁺Vβ8⁺ T cells following co-culture with effector CD8⁺ T cells and CD4⁺Vβ8⁻ T cells leads to a reduced ratio of CD4⁺Vβ8⁺ to CD4⁺Vβ8⁻ that could be precisely enumerated by two color FACS. This method of measuring specific cytotoxicity permitted analysis of co-cultured effector and target cells for greater than 24 hours. To demonstrate that the FACS CTL method gives quantitative results comparable to conventional ⁵¹Cr release assays, a murine alloantigen specific CTL line, L3 cells (H-2b), their specific CTL activity to the specific targets, P815 cells (H-2d), and non-specific targets EL4 cells (H-2b) were simultaneously tested in both FACS and ⁵¹Cr release assays. Similar CTL activities were obtained using both assays at effector/target ratio from 5 to 1.25 (FIGS. 8A and 8B and Table 4). By FACS assay the ratio of H-2⁺ to H-2⁻ targets clearly decreased in cultures containing anti-H-2^{d} killers (FIG. 8B) compared to cultures without effector cells (FIG. 8A).

Using the FACS CTL assay the specificity of the CD8⁺Vβ8-cell lines was tested next. In particular, the ability of CD8⁺ T cell line cells initially induced by CD4⁺Vβ8⁺ cells to specifically kill CD4⁺Vβ8⁺ targets was investigated. The results using four independent CD8⁺Vβ8⁻ T cell lines (CTL-1 to 4) co-cultured for 24 hours with target populations containing varying numbers of CD4⁺Vβ8⁺ and CD4⁺Vβ8⁻ T cells in five separate experiments is shown in Table 5. The target cell populations were derived from cultures of CD4⁺ cells triggered by varying ratios of the superantigens SEB to SEE to generate cell lines with varying numbers of Vβ8⁺ and Vβ8⁻ (containing Vβ11⁺) cells. The differences of the ratio of Vβ8⁺ versus Vβ8⁻ T cells in cultures with and without effectors reflects the specific deletion of Vβ8⁺ cells. The ratio of Vβ11⁺/Vβ11⁻ cells as a specificity control was simultaneously measured. As shown, CD8⁺Vβ8⁻ T cells deleted the CD4⁺Vβ8⁺ T cells but not CD4⁺Vβ8⁻ T cells. This was shown by the decreased ratio of CD4⁺Vβ8⁺/CD4⁺Vβ8⁻ (Vβ8⁺/Vβ8⁻), and as expected, the increased ratio of CD4⁺Vβ11⁺/CD4⁺Vβ11⁻ (Vβ11⁺/Vβ11⁻) in target cells. One explanation of the preceding experiments was that specificity was not only a function of the TCR Vβ used by the target T cells but was influenced by the different superantigens used to generate the targets.

TABLE 4
A Comparison of CTL Activity of L3 Cells in Both FACS
Assay and ⁵¹Cr Release Assay

Specific cytotoxicity (percentage)
E:T ratio

| Target Cells | 5:1 | 2.5:1 | 1.25:1 |
|---|---|---|---|
| FACS Assay^{a} | | | |
| P815 + EL4 | 75 | 65 | 43 |
| ⁵¹CR Release Assay^{b} | | | |
| P815 | 72 | 57 | 44 |
| EL4 | 1.5 | 0.6 | 1.0 |
| ^{a}In the FACS assay, specific targets P815 and nonspecific targets EL4 were mixed in a 2:1 ratio, then L3 cells were added to | | | |
| # the mixed targets at different E:T ratio. Targets without L3 cells served as control. After 4 hr of incubation, the cells were | | | |
| # stained with F-34.1.2 and APC-53-6.72, and the data was analyzed as described in Experimental Procedures. | | | |
| ^{b}In the ⁵¹CR release assay, P815 and EL4 cells were labeled with ⁵¹Cr: then L3 | | | |
| # cells were added to both targets at different E:T ratio separately. After 4 hr of incubation, the supernatant was | | | |
| # harvested, the radioactivity was counted and specific cytolysis was calculated as described in Experimental Procedures. | | | |

TABLE 5
TCR Vβ8-Specific CD8⁺T Cells Selectively Deleted CD4⁺Vβ8⁺T Cells but Not
CD4⁺Vβ8⁻T Cells in Mixed T Cell Cultures

| Targets | | | | | Ratio of CD4⁺ Vβ8⁺/ | | | | |
|---|---|---|---|---|---|---|---|---|---|
| Experiment | | | | | CD4⁺ Vβ8⁻ | | | Ratio of | |
| CD8⁺ | | | CD8⁺ | | | Plus | Percentage | CD4⁺ Vβ11⁺ | |
| Number | | | Effector | E:T | | CD8⁺ | of specific | CD4⁺ Vβ11⁻ | |
| effectors | Cells | Stimulant^{a} | Cells | Ratio | Control | effectors | deletion | Control | Plus |
| 1 | CD4⁺ | SEB(1) plus SEE (0.1) | CTL-1 | 4:1 | 0.47 | 0.34 | 27.6 | 0.14 | 0.16 |
| | | | | | | | | | (0.16)^{b} |
| 2 | CD4⁺ | SEB(1) plus SEE (0.1) | CTL-2 | 2:1 | 0.31 | 0.24 | 22.5 | 0.17 | 0.18 |
| | | | | | | | | | (0.18) |
| | | | | 1:1 | | 0.23 | 25.8 | | 0.18 |
| | | | | | | | | | (0.18) |
| 3 | CD4⁺ | SEB(1) plus SEE (0.1) | CTL-2 | 2:1 | 0.32 | 0.24 | 25.0 | 0.18 | 0.19 |
| | | | | | | | | | (0.19) |
| | | | | 1:1 | | 0.23 | 28.1 | | 0.19 |
| | | | | | | | | | (0.19) |
| 4 | CD4⁺ | SEB(1) plus SEE (0.025) | CTL-2 | 2:1 | 3.20 | 2.00 | 37.5 | 0.04 | 0.05 |
| | | | | | | | | | (0.05) |
| | | | | 1:1 | | 1.94 | 39.3 | | 0.06 |
| | | | | | | | | | (0.05) |
| 5 | CD4⁺ | SEB(1) plus SEE (0.025) | CTL-3 | 2:1 | 4.60 | 3.37 | 26.7 | — | — |
| | | | | 1:1 | | 3.54 | 23.0 | — | — |
| | | | CTL-4 | 2:1 | | 3.57 | 25.8 | — | — |
| | | | | 1:1 | | 3.59 | 22.0 | — | — |

Target T cells were purified CD4⁺ T Cells from BALB/c mice stimulated with SEB and SEE, and TCR Vβ8-specific CD8⁺ Vβ8⁻
CTL lines were prepared as described in Experimental Procedures. Target cells were cultured alone (control) or mixed
with the CD8⁺ CTL lines (plus CD8⁺ effectors) at the indicated ratios and cultured for 24 hr in the presence of IL-2(10
U/ml) . The cells were then divided into two tubes, washed, and stained with F-53.6.72 and biotin-F23.1 or biotin-RR-
3.15 and analyzed as described in FIGS. 7A and 7B. For data analysis, the CD8⁺T cells were gated out and the data
were expressed as the ratio of CD4⁺ Vβ8⁺ T cells versus CD4⁺ Vβ8⁻ T cells (Vβ8⁺/Vβ⁸⁻) or CD4⁺ Vβ11⁺ T cells versus CD4⁺
Vβ11⁻ cells (Vβ11⁺/Vβ11⁻).
The percentage of the specific deletion of CD4⁺ Vβ8⁺ T cells was calculated as:
$\frac{V {β8}^{+} / V {β8}^{-} of control - V β {8}^{+} / V β {8}^{-} of experimental}{V β {8}^{+} / V β {8}^{-} of control} \times 100 \%$
^{a}To obtain different ratios of CD4⁺ Vβ8⁺/CD4⁺Vβ8⁻ of target populations in SEB/SEE cultures, same amount of SEB (1 μm/ml)
and different amount of SEE (μg/ml) were added in CD4⁺ T cell cultures as indicated.
^{b}The values in parenthesis are the theoretical calculated values of the CD4⁺ Vβ11⁺/CD4^{+V}β11⁻ ratio if CD4⁺Vβ11⁺ T cells in
the targets were not deleted by the CD8⁺ T cells.

To exclude this possibility, the observation that SEB activates Vβ7⁺ and Vβ17⁺ as well as Vβ8⁺ T cells in BALB/c mice (Marrack and Kappler, 1990) was taken advantage of, and SEB was used to generate both CD4⁺Vβ8⁺ and CD4⁺Vβ8-targets. These targets were derived either from purified total CD4⁺ T cells stimulated by SEB (Table 6. Exp.1 and 2), or from purified CD4⁺Vβ8⁺ and purified CD4⁺Vβ8⁻ T cells stimulated by SEE separately, and mixed at a Vβ8⁺/Vβ8⁻ ratio of 1:1 before the test (Table 6. Exp.3.). Again, only TCR Vβ8 specific cytotoxicity was observed. These data also rule out SEE carryover as a mechanism of TCR Vβ8 specific cytotoxicity, because if SEE carryover was involved in the CD8⁺ T cell-mediated killing, there should have been comparable killing in both Vβ8⁺ and Vβ8⁻ populations. This was not observed, even though the Vβ8⁻ T cell population contains SEE-reactive cells including those expressing Vβ7 and Vβ17. Taken together, these data strongly suggest that the CD8⁺ T cell line initially induced by CD4⁺Vβ8⁺ cells differentiate into killer cells which specifically lyse CD4⁺Vβ8⁺ targets.

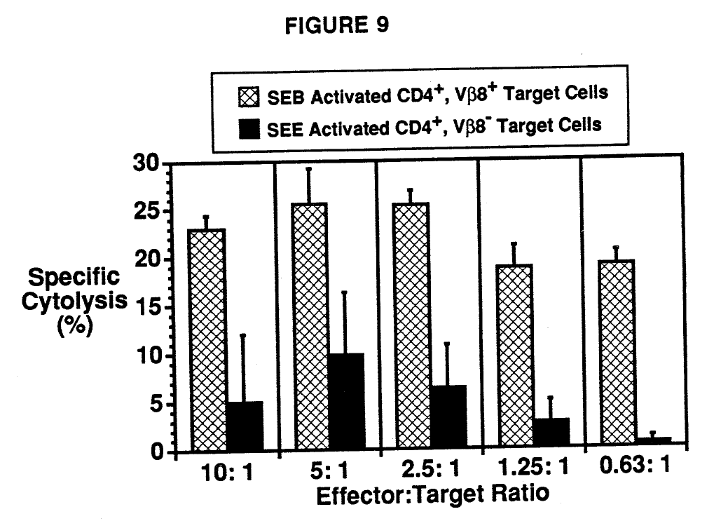

However, because the FACS assay quantitatively measures the fraction of Vβ8⁺ cells present after 24 hours of culture, one interpretation of the above data was that the Vβ8⁺ cells were not killed but merely suppressed ingrowth. To directly determine if cell lysis was occurring CD8⁺ effectors were co-cultured with the CD4⁺Vβ8⁺ or CD4⁺Vβ8⁻ targets separately at varying effector/target ratios for 12 hours and assayed cell lysis by measuring specific ⁵¹Cr release. As shown in FIG. 9, four independent CD8⁺ T cell lines specifically lysed the CD4⁺Vβ8⁺ T cell targets.

TABLE 6
TCR Vβ8-Specific CD8⁺ T Cells Selectively
Deleted CD4⁺ Vβ8⁺ T Cells but
Not CD4⁺ Vβ8⁻ T Cells in
Mixed T Cell Cultures

| Percentage of | | | Ratio of CD4⁺ | | | |
|---|---|---|---|---|---|---|
| specific | | | Vβ8⁺/CD4⁺ Vβ8⁻ | | | |
| Experiment | | CD8⁺ Effector | | | Plus CD8⁺ | deletion |
| Number | Target Cells | Cells | E:T | Ratio Control | effectors | (CD4⁺Vβ8⁺) |
| 1 | CD4⁺ | CTL-3 | 2:1 | 8.20 | 5.50 | 28.9 |
| | | CTL-4 | 2:1 | | 6.00 | 26.8 |
| 2 | CD4⁺ | CTL-3 | 2:1 | 8.49 | 6.26 | 26.3 |
| | | CTL-4 | 2:1 | | 6.23 | 26.8 |
| 3 | CD4⁺ Vβ8⁺Vβ8⁻ | CTL-3 | 2:1 | 1.05 | 0.71 | 32.4 |
| | | | 1:1 | | 0.72 | 31.4 |
| | | CTL-4 | 2:1 | 1.05 | 0.73 | 30.5 |
| | | | 1:1 | | 0.74 | 29.5 |

The experiments were performed as in Table 5. Target cells were stimulated with SEB only. To obtain different initial
# Vβ8⁺/Vβ8⁻ ratios, total purified CD4⁺ T cells were stimulated with SEB (1 μg/ml)
# (Experiments 1 and 2) or purified CD4⁺ Vβ8⁺ and CD4⁺ Vβ8⁻ T cells were
# stimulated with SEB (1 μg/ml) separately and mixed in a 1:1 ratio prior to use as targets (Experiment 3).

The Vβ8-Specific in vitro Cytotoxicity Mediated by CD8⁺ T Cells is Dependent on B₂ m-Associated Molecules.

Because CD8⁺ cytotoxic T cells usually recognize antigens associated with MHC class I molecules the ability of CD4⁺Vβ8⁺ target cells derived from β₂m-/- mice to be lysed by the Vβ8 specific CD8⁺ CTL was investigated. Thus, Vβ8 specific CTLs derived from B6 mice were added to CD4⁺Vβ8⁺ and CD4⁺Vβ8⁻ targets from B6 mice or from β₂m-/- mice, and deletion of CD4⁺Vβ8⁺ cells was assayed by FACS. No deletion of CD4⁺Vβ8⁺ cells were observed when targets were derived from the β₂m-/- mice (Table 7). These same β₂m-/- targets were susceptible to killing in a mitogen (Con A) induced MHC class I independent assay. Furthermore, deletion of CD4⁺Vβ8⁺ T cells was readily observed in the control B6 targets. (Table 7). Taken together, these data show that the Vβ8 specific in vitro cytotoxicity mediated by CD8⁺ T cells is dependent on B2 microglobulin-associated molecules.

TABLE 7
CD4⁺ Vβ8+T Cells from β₂m^{-/-} Mice
Can Not Be Killed by Syngenic TCR Vβ8-Specific CTLs

Ratio of CD4⁺ Vβ8⁺/CD4⁺ Vβ8⁻

| | | | | | Percentage |
|---|---|---|---|---|---|
| Expeeriment | CD4+ | | | | of |
| Number | Target | E:T | | CD8³⁰ | specific |
| (CD4^{+Vβ8}⁺) | Cells | Ratio | Control | effectors | deletion |
| 1 | C57BL/6 | 2:1 | 0.33 | 0.26 | 21.2 |
| | | 1:1 | | 0.27 | 18.2 |
| | β₂m^{-/-} | 2:1 | 0.92 | 0.89 | 3.3 |
| | | 1:1 | | 1.00 | -8.6 |
| 2 | C57BL/6 | 2:1 | 0.45 | 0.32 | 28.9 |
| | | 1:1 | | 0.33 | 26.7 |
| | β₂m^{-/-} | 2:1 | 0.60 | 0.58 | 3.3 |
| | | 1:1 | | 0.59 | 1.7 |
| CD4+ Vβ8⁺ and CD4^{+ Vβ8-} targets from B6 and B₂m^{-/-mice were stimulated} | | | | | |
| # with SEB, and served as targets. TCR Vβ8-specific CTLs were prepared from B6 mice as described in Experimental Procedures. | | | | | |

The Vβ8 Specific in vitro Cytotoxicity Mediated by CD8⁺ T Cells is Not Blocked by Antibody to MHC Class I-a Molecules.

The next set of experiments were designed to identify which B₂ microglobulin-associated molecules were involved in the Vβ8 specific in vitro cytotoxicity mediated by CD8⁺ T cells. Because β₂ microglobulin molecules are known to be associated with MHC class I, antibodies known to block classical MHC class I restricted allogeneic CTL responses were tested for their ability to inhibit the CD8⁺ cells mediating Vβ8 specific killing of syngeneic CD4⁺ cells. For these experiments the M1/42.39 antibody which is a rat anti mouse H-2 monoclonal antibody known to be specific for all H-2 haplotypes including H-2Kd and H-2Dd and H-2Ld MHC class I antigens (Stallcup et al., 1981) was used. The first set of experiments demonstrated the effectiveness of the M1/42.39 antibody in blocking allospecific cytotoxicity. L3 cells which are H-2b CD8+allospecific killers and efficiently lyse allogeneic P815 (H-2d) targets were used and the cytotoxicity in the presence or absence of the M1/42.39 antibody or control normal rat Ig was tested. These experiments showed that M1/42.39 but not the control rat Ig markedly inhibited the lysis of P815 allogeneic targets over a range of antibody concentration from 800 μg/ml to 25yg/ml. Next, the effectiveness of the M1/42.39 antibody in blocking the CD8⁺ cells mediating Vβ8 specific killing of syngeneic CD4⁺ cells in BALB/c mice (H-2^{d}) was tested. Vβ8 specific CD8⁺ killers were generated as before and cytotoxicity of CD4⁺Vβ8⁺ targets tested in the presence or absence of M1/42.39 or control rat Ig. In four separate experiments, the M1/42.39 antibody, although highly efficient in blocking conventional CTL directed against H-2^{d} at 25 μg/ml, does not block the Vβ8 specific killing of CD4⁺ targets at 200 μg/ml, even though the targets express the H-2^{d} MHC class I alleles recognized by the Ml/42.39 monoclonal antibody. These data suggest that the MHC class I-a determinants recognized by M1/42.39 antibody are not involved in the cytotoxicity mediated by the Vβ8 specific CD8⁺ killer cells. These data are consistent with the analysis of Vβ specific CD8⁺ killer cells in man which are not inhibited by the antibody W6/32 known to be reactive with all known MHC class I-a molecules (Ware et al., 1994).

The Vβ8 Specific in vitro Cytotoxicity Mediated by CD8⁺ T Cells is Efficiently Blocked by Antiserum to the MHC Class I-b Molecule, Qa-1.

The above data demonstrated that although the Vβ8 specific in vitro cytotoxicity of CD4⁺ cells mediated by CD8⁺ T cells is dependent on B₂ microglobulin-associated molecules, antibody to the classical MHC class I-a molecules did not block the cytotoxicity. These data raised the possibility that other B₂ microglobulin-associated molecules, including MHC class I-b molecules might be involved as target structures for the Vβ specific killers. Moreover, because the MHC class I-b molecule, Qa-1, has previously been implicated as defining populations of regulatory cells important in immune suppression (Eardley et al., 1978), Qa-1 might be involved as a structure expressed on CD4⁺ cells in the CD8⁺ T cell mediated Vβ specific killing. To test this idea, a series of blocking studies were performed using well characterized sera which initially defined the Qa-1a and Qa-1b alleles. In initial experiments the Qa-1b but not the Qa-1a determinant was shown to be expressed on the CD4⁺ target cells derived from BALB/c mice. The antisera to Qa-1a and Qa-1b was also shown to not block the killing of P815 target cells by CD8⁺ anti-MHC class I-a allogeneic killer L3 cells. These data indicated that the Qa-1a and Qa-1b antisera were not nonspecifically capable of blocking cell mediated cytotoxicity. Next, the effectiveness of the anti-Qa-1b antiserum in blocking the CD8⁺ cell mediated Vβ8 specific killing of syngeneic CD4⁺ cells in BALB/c mice was tested and anti-Qa-1a antiserum as a specificity control was used. Vβ8 specific CD8⁺ killers were generated as before and cytotoxicity of CD4⁺Vβ8⁺ targets tested in the presence or absence of Qa-1a and Qa-1b antisera (Table 8). In 6 separate experiments Vβ specific cytotoxicity was shown to be blocked by the specific Qa-1b antiserum but not by Qa-1a antiserum.

TABLE 8
Antimurine Qa-1b but Not Anti-Qa-la Antisera Blocked TCR Vβ8-Specific Killing in BALB/c Mice*

| | Ratio of CD4⁺ Vβ8^{+/CD4}⁺ Vβ8⁻ | | | | | | Percentage |
|---|---|---|---|---|---|---|---|
| | | Plus CD8⁺ effectors | | | Percentage of | Percentage | of Qa-1b |
| Experiment | | | | | Vβ-specific | of | specific |
| Number | Control | Minus Ab | Plus αQa-1^{a} | Plus αQa-1^{b} | deletion | blocking | blocking |
| | 0.32 | 0.25 | | | 21.9 | — | |
| 1 | | | 0.26 | | 19.0 | 13.2 | |
| | | | | 0.33 | 0 | 100 | 86.8 |
| | 0.23 | 0.16 | | | 30.4 | — | |
| 2 | | | 0.17 | | 26.1 | 14.1 | |
| | | | | 0.24 | 0 | 100 | 85.9 |
| | 0.88 | 0.52 | | | 40.9 | — | |
| 3 | | | 0.55 | | 37.5 | 8.3 | |
| | | | | 0.74 | 15.9 | 61.1 | 52.8 |
| | 0.38 | 0.24 | | | 36.8 | — | |
| 4 | | | 0.25 | | 34.2 | 7.1 | |
| | | | | 0.34 | 10.5 | 71.5 | 64.4 |
| | 0.58 | 0.32 | | | 44.8 | — | |
| 5 | | | | 0.34 | | 41.4 | 7.6 |
| | | | | 0.59 | 0 | 100 | 92.4 |
| | 0.76 | 0.54 | | | 28.9 | — | |
| 6 | | | 0.53 | | 30.3 | 0 | |
| | | | | 0.73 | 3.9 | 86.5 | 86.5 |
| *TCR Vβ8-specific CD8^{+T cells and CD4}⁺ Vβ8⁺, CD4⁺ Vβ8⁻ targets were prepared and experiments were performed as described in Table 6. Anti-Qa-1 sera were added at a final dilution of 1:10 to the targets and incubated at | | | | | | | |
| # room temperature for 30 min before mixed with effector cells. Percentage of blocking of killing was calculated as: {[percentage of killing of control group (without antisera) minus percentage of killing of experimental group (with antisera)]/percentage of killing of control group} × 100%. | | | | | | | |

In the present studies CD8⁺ T cells were shown to participate in the in vivo regulation of CD4⁺Vβ8⁺ T cells following SEB administration. Previous work (Kawabe and Ochi, 1991) that showed a deletion of 30-40% of CD4⁺Vβ8⁺ cells 7-14 days after a single injection of SEB in mice was first confirmed. The downregulation of CD4⁺Vβ8⁺ T cells below baseline was shown not to be observed in mice depleted of CD8⁺ cells by treatment with monoclonal anti-CD8 antibody or in CD8⁺ T cell deficient β₂m-/- mice. Moreover, the current studies show that following SEB administration, splenic and lymph node CD8⁺ T cells preferentially recognizing CD4⁺Vβ8⁺ T cells are generated and can be specifically expanded in vitro. These CD8⁺ T cells are cytotoxic to autologous CD4⁺Vβ8⁺ T cells but not to autologous CD4⁺Vβ8⁻ T cells. Furthermore, these studies demonstrated that this autologous TCR Vβ specific cytotoxicity is dependent on recognition of B2 microglobulin-associated molecules and is inhibited by antiserum specific for Qa-1 molecules but not by antibody to classical MHC class I-a molecules. Together these data support the idea that the specificity of immune regulation is mediated by specific recognition by CD8⁺ T cells of TCR Vβ chains or peptides derived from TCR Vβ chains bound to Qa-1 MHC class I-b molecules expressed on the surface of autologous CD4⁺ T cells.

The evidence that CD8⁺ T cells arise which are specific for CD4⁺ cells expressing particular Vβ TCRs was demonstrated by showing that CD8⁺ T cells obtained by stimulation with SEB activated CD4⁺Vβ8⁺ T cells only kill SEB activated CD4⁺Vβ8⁺ T cells but not SEB activated CD4⁺Vβ8⁻ T cells even though SEB activates Vβ7⁺ and Vβ17⁺ T cells as well as Vβ8⁺ T cells in BALB/c mice. The destruction of CD4⁺ cells by a conventional ⁵¹Cr release assay was assayed as well as by measuring the change in the ratio of Vβ8⁺/Vβ8⁻cells induced by CD8⁺ effector cell in mixed T cell culture by FACS. The killing observed is not related to T cell activation alone because Vβ8⁺ and Vβ8⁻targets cells were both activated. Further, the killing is not dependent on the particular superantigen used because Vβ8⁻ T cells activated either by SEE or SEE were not killed whereas Vβ8⁺ cells activated by SEB were killed.

Because CD8⁺ T cells usually recognize target cells expressing particular MHC Class I/peptide complexes, the Vβ specific CD8⁺ T cells recognize MHC class I molecules complexed to peptides derived from TCR Vβ chains on target T cells. The hypothesis that the self TCR antigens must either be associated with or presented by MHC class I molecules is unequivocally supported by the findings that target cells derived from β₂m-/- mice which are devoid of both MHC class I-a and MHC class I-b molecules do not function as targets for the Vβ specific CD8⁺ effector cells. It is of interest to point out that due to the limited polymorphism and tissue-restricted expression of MHC class I-b molecules, they may be highly appropriate for the presentation of endogenous, self peptides (Stroynowski, 1990; Joyce et al., 1994; Aldrich et al., 1994). The data obtained from the current experiments that the TCR Vβ specific killing mediated by the CD8⁺ T cells could be blocked by anti-Qa-1 antiserum but not anti- MHC class I-a antibody strongly indicate that Qa-1 molecules are the major self TCR peptide presenting molecules in this particular T-T cell interaction. Since anti-Qa-1 serum only blocked about 70-80% of the TCR Vβ specific killing in this study, the possibility that other MHC Class I-b molecules are also involved in this cellular event could not be excluded. On the other hand, the finding that a monoclonal anti-Class I-a antibody, M1/42.39, did not inhibit Vβ restricted cytotoxicity, is against a role of Class 1-a MHC in this system. The results from EXAMPLE 1 that anti human MHC class I-a antibody W6/32 did not block Vβ specific cytotoxicity mediated by human CD8⁺ T cells further support the hypothesis that non-polymorphic MHC class I-b molecules are the presenting molecules involved. Moreover, studies in EAE also suggest that non-polymorphic MHC class I-b molecules can be important in immune regulation. Sun et al (Sun et al., 1988) isolated a CD8⁺ T cell line from EAE rats induced by the encephalitogenic CD4⁺ T cell line “S1”. This autologous, anti-S1, CD8⁺ T cell line proliferated when cultured with S1 cells but not when cultured with MBP presented by classical APCs. Moreover, the anti-S1, CD8⁺ T cells were cytotoxic to S1 cells and the specific proliferation of the CD8⁺ T cells to S1 cells could not be blocked by anti-MHC class I-a antibody. In addition, it is interesting that studies of regulatory T suppressors indicated that the CD4⁺ inducers of CD8⁺ suppressors expressed high levels of the Qa-1 molecules and if the inducer population was depleted of Qa-1 expressing T cells, they lost the capacity of inducing T suppressors (Eardley et al., 1978). The results of this study, viewed in this light, indicate that Qa-1 molecules play a role in the control of immune responses by presenting self TCR peptides to regulatory CD8⁺ T cells. The in vitro experiments described here show that the CD8⁺ effector cells killed the specific CD4⁺ targets at low E/T ratios. However, the maximum fraction of CD4⁺Vβ8⁺ cells lysed was only approximately 30%. There are several possible explanations for such partial killing of target T cells in vitro. First, the expression of Qa-1/TCR Vβ target structure may not be present in sufficient quantity on all targets and limit the number of cells killed. Second, Qa-1⁺ and Qa-1⁻ CD4⁺ T cells with differential susceptibility to killing may emerge following antigen activation. Third, it has been shown that most of the CD4⁺Vβ8⁺ T cells that survived 10-14 days after SEB administration in vivo were anergic (Rellahan et al., 1990; Kawabe and Ochi, 1990). This raises the possibility that the state of anergy of CD4⁺ T cells is correlated to their susceptibility to the lysis. In addition, it is known, that the interactions between cytotoxic T cells and their specific targets is not only dependent on cognitive recognition between TCR and specific antigen/MHC complexes, but is also dependent on the interactions of adhesive molecules including CD2, VLA, LFA1 and LFA3 (Williams and Barclay, 1988; Staunton et al., 1988; Shimizu et al., 1990). The expression of these molecules may also vary with T cell activation and limit killing mediated by CD8⁺Vβ specific effector cells.

It is of interest to relate the in vitro cytotoxicity observed to the SEB induced delayed, CD8⁺ T cell-dependent deletion (4-21 days after SEB administration) of CD4⁺Vβ8⁺ T cells observed in vivo (FIGS. 7A and 7B and Table 3). The mechanisms of this CD8⁺ T cell dependent CD4⁺ T cell deletion in vivo is most likely complex. It can be envisioned that populations of Vβ8 specific CD8⁺ T cells are initially stimulated by SEE reactive CD4⁺Vβ8⁺ T cells to grow and differentiate. These Vβ8 specific CD8⁺ T cells secrete a variety lymphokines which directly or indirectly are responsible for the death of activated Vβ8 expressing CD4⁺ T cells. In addition, the deletion of CD4⁺ T cells could result from the direct cytotoxic T effector function of the Vβ8 specific CD8⁺ killer cells that was documented in vitro. The relationship between the current in vitro and in vivo data is also highlighted by the present experiments using B₂m-/- mice. In these CD8⁺ T cell deficient animals there is no delayed deletion of CD4⁺Vβ8⁺ T cells below baseline (Table 3) and, perhaps as importantly, CD4⁺Vβ8⁺ targets cells from the β₂m-/- mice are not lysed by CD8⁺ effector cells derived from syngeneic β₂m₊ mice (Table 7).

The idea that regulatory CD8⁺ T cells which recognize TCR target structures expressed on CD4⁺ T cells are important in the control of immune responses in vivo is supported by several other lines of investigation. First, in studies of EAE in mice, rats and human it has been shown that encephalitogenic T cells expressing specific Vβ segments can induce autoregulatory T cells (Sun et al., 1988; Cohen and Weiner, 1988; Lider et al., 1988; Lohse et al., 1989; Zhang et al., 1993). Moreover, TCR peptides derived from encephalitogenic CD4⁺ T cells could protect animals from the development of EAE in vivo (offner et al., 1991; Vandenbark et al., 1989; Howell et al., 1989). Further, it has been shown that CD8⁺ CTL emerge in rats that have recovered from EAE induced by an encephalitogenic CD4⁺ cell line (Sun et al., 1988). These CD8⁺ CTL are specific for the inducing T cell line and, importantly, efficiently neutralize the encephalitogenic functions of the inducing cells in vivo. In addition, it has recently been shown that in the EAE model in mice regulatory CD4⁺ T cells also emerge which downregulate the antigen specific immune response mediated by CD4⁺Vβ8.2⁺ T cells (Kumar and Sercarz, 1993). However, even these regulatory CD4⁺ cells were shown to be CD8⁺ T cell dependent. Thus, immunoregulatory networks involving regulatory CD4⁺ and CD8⁺ T cells both recognizing TCR Vβ segments ultimately control the immune response in EAE. Furthermore, Kimura and Wilson (Kimura and Wilson, 1984) showed that during the development of graft-versus-host disease (GvH) in rats CD8⁺ CTL derived from A/B F1 strain rats emerge which specifically kill A strain anti-MHC B CD4⁻ T blasts but do not kill A strain anti-MHC C CD4⁺ T blasts. These cytotoxic CD8⁺ T cells do kill anti-MHC B CD4⁺ T blasts from a third party strain C. Thus, like the CD8⁺ CTL described in this study, these CTL recognize a common target structure expressed on CD4⁺ T blasts derived from MHC A or C strain which is most likely the TCRs specific for MHC B alloantigen. Interestingly, these CTL are not restricted by classical polymorphic MHC class I-a molecules. This kind of CTL might be responsible for the resistance of GvH disease induced by previous inoculation of T cells from one of the parental strain in Fl rats in vivo.

The results described in this study are not confined to Vβ8 in mice and experimental support for this notion is present in EXAMPLE 1. There, it is demonstrated that human CD8⁺ T clones specific for TCR Vβs can be induced in vitro by superantigen activated autologous CD4⁺ T cell clones. These human CD8⁺ T clones specifically recognize and kill autologous clones of CD4⁺ target cells expressing particular Vβ TCRs. This killing is not inhibited by antibody to human MHC class I-a molecules. Thus, the TCR Vβ specific cytotoxicity mediated by CD8⁺ T cells described here represent a more general phenomenon and plays important physiological roles in immunoregulation and in control of autoimmunity.

### REFERENCES

1. Aldrich, C. J., DeClousc, A., Woods, A. S., Cotter, R. J., Woloski, M. J. and Forman, J. (1994). Identification of a TAP-dependent header peptide recognized by alloreactive T cells specific for a Class I-b antigen. Cell 79, 649-659.

2. Bloom, B. R., Modlin, R. L. and Salgame, P. (1992a). Stigma variations: observations on suppressor T cells and leprosy. Annual Rev. Immunol. 10, 453-88.

3. Bloom, B. R., Salgame, P. and Diamond, B. (1992b). Revisiting and revising suppressor T cells. Immunology Today 13, 131-6.

4. Boehme, S. A. and Lenardo, M. J. (1993). Propiocidal apoptosis of mature T lymphocytes occurs at S phase of the cell cycle. Eur. J. Immunol. 23,1552-1560.

5. Boyse, E. A., Flaherty, L., Stockert, E. and Old, L. J. (1972). Histocompatibility attributable to genes near H-2 that are not revealed by hemagglutination or cytotoxicity test. Transplantation 13, 431-432.

6. Cohen, I. R. and Weiner, H. L. (1988). T-cell vaccination. Immunol. Today 9, 332-335.

7. Dangl, J. L., Parks, D. R., Oi, V. T. and Herzenberg, L. A. (1982). Rapid isolation of cloned isotype switch variants using fluorescence activated cell sorting. Cytometry 2, 395-401.

8. Dorf, M. E. and Benacerraf, B. (1984). Suppressor cells and immunoregulation. Ann. Rev. Immunol. 2, 127-57.

9. Eardley, D. D., Hugenberger, J., McVay-Boudreau, L., Shen, F. W., Gershon, R. K. and Cantor, H. (1978). Immunoregulatory circuits among T-cell sets. I. T-helper cells induce other T-cell sets to exert feedback inhibition. J. Exp. Med. 147, 1106-15.

10. Fellows, M., Kamoun, M., Gresser, I., and Bono, R. (1979). Enhanced expression of HLA antigens and beta2-microglobulin on interferon treated human lymphoid cells. Eur. J. Immunol. 9,446.

11. Friedman, S. M., Hunter, S. B., Irigoyen, O. H., Kung, P. C., Goldstein, G. and Chess, L. (1981). Functional analysis of human T cell subsets defined by monoclonal antibodies. II. Collaborative T-T interactions in the generation of TNP-altered-self-reactive cytotoxic T lymphocytes. J. Immunol. 126, 1702-5.

12. Gaur, A., Haspel, R., Mayer, J. P. and Fathman, C. G. (1993). Requirement for CD8⁺ cells in T cell receptor peptide-induced clonal unresponsiveness. Science 259, 91-4.

13. Gaur, A., Ruberti, G., Haspel, R., Mayer, J. P. and Fathman, C. G. (1993). Requirement for CD8⁺ cells in T cell receptor peptide-induced clonal unresponsiveness. Science 259, 91-4.

14. Glasebrook, A. and Fitch, F. W. (1979). T cell lines which cooperate in generation of specific cytolytic activity. Nature 278, 171-173.

15. Gonzalo, J. A., Baixeras, E., Gonzalez-Garcia, A., George-Chandy, A., Van-Rooijen, N., Martinez, C. and Kroemer, G. (1994). Differential in vivo effects of a superantigen and an antibody targeted to the same T cell receptor. Activation-induced cell death vs passive macrophage-dependent deletion. J. Immunol 152, 1597-1608.

16. Goodman, J. W. and Sercarz, E. E. (1983). The complexity of structures involved in T-cell activation. Ann. Rev. Immunol. 1, 465-98.

17. Green, R. D., Flood, P. M. and Gershon, R. K. (1983). Immunoregulatory T-cell pathways. Ann. Rev. Immunol. 1, 439-63.

18. Howell, M. D., Winters, S. T., Olee, T., Powell, H. C., Carlo, D. J. and Brostoff, S. W. (1989). Vaccination against experimental allergic encephalomyelitis with T cell receptor peptides. Science 246, 668-670.

19. IFBN Multiple Sclerosis Study Group (1993). Interferon beta-lb is effective in relapsing-remitting multiple sclerosis. I. Clinical results of a multicenter, randomized, double-blind placebo-controlled trial. Neurology 43, 655.

20. Jandinski, J., Cantor, H., Tadakuma, T., Peavy, D. L. and Pierce, C. W. (1976). Separation of helper T cells from suppressor T cells expressing different Ly components. I. Polyclonal activation: suppressor and helper activities are inherent properties of distinct T-cell subclasses. J. Exp. Med. 143, 1382-90.

21. Jerne, N. K., in Harvey Lecture Series (Academic Press, New York., 1975), pp. 93-110.

22. Jiang, H., Zhang, S. I. and Pernis, B. (1992). Role of CD8⁺ T cells in murine experimental allergic encephalomyelitis. Science 256, 1213-5.

23. Joyce, S. T., P. Angeletti, R. H. Nathenson, S. G. Stroynowski, I. (1994). A nonpolymorphic major histocompatibility complex class Ib molecule binds a large array of diverse self-peptides. J. Exp. Med. 179, 579-588.

24. Kawabe, Y. and Ochi, A. (1990). Selective anergy of Vβ8⁺ CD4⁺ T cells in Staphylococcus enterotoxin B-primed mice. J. Exp. Med. 172, 1065-1070.

25. Kawabe, Y. and Ochi, A. (1991). Programmed cell death and extrathymic reduction of Vbeta8⁺ CD4⁺ T cells in mice tolerant to Staphylococcus aureus enterotoxin B. Nature 349, 245-8.

26. Kimura, H. and Wilson, D. B. (1984). Anti-idiotypic cytotoxic T cells in rats with graft-versus-host disease. Nature 308, 463-4.

27. Koh, D. -R., Fung-Leung, W. -P., Ho, A., Gray, D., Acha-Orbea, H. and Mak, T. -W. (1992). Less mortality but more relapses in experimental allergic encephalomyelitis in CD8-/- Mice. Science 256, 1210-3.

28. Koide, J. and Engleman, E. G. (1990). Differences in surface phenotype and mechanism of action between alloantigen-specific CD8⁺ cytotoxic and suppressor T cell clones. J. Immunol. 144, 32-40.

29. Kumar, V. and Sercarz, E. E. (1993). The involvement of T cell receptor peptide-specific regulatory CD4⁺ T cells in recovery from antigen-induced autoimmune disease. J. Exp. Med. 178, 909-16.

30. Lamb, J. R. and Feldman, M. (1982). A human suppressor T cell clone which recognizes an autologous helper cell clone. Nature 300, 456-8.

31. Lederman, S., Yellin, M. J., Krichevsky, A., Belko, J., Lee, J. J. and Chess, L. (1992). Identification of a novel surface protein on activated CD4⁺ T cells that induces contact-dependent B cell differentiation (help). J. Exp. Med. 175, 1091-101.

32. Lenardo, M. J. (1991). Interleukin-2 programs mouse alpha beta T lymphocytes for apoptosis. Nature 353, 858-861.

33. Lider, O., Reshef, T., Beraud, E., Ben-Nun, A. and Cohen, I. R. (1988). Anti-idiotypic network induced by T cell vaccination against experimental autoimmune encephalomyelitis. Science 239, 181-183.

34. Lindahl, P., Leary, P., and Gresser, I. (1974). Enhancement by interferon of the expression of histocompatibility antigens of mouse lymphoid cells in vitro. Eur. J. Immunol. 4,779.

35. Lohse, A. W., Mor, F., Karin, N. and Cohen, I. R. (1989). Control of experimental autoimmune encephalomyelitis by T cells responding to activated T cells. Science 244, 820-822.

36. Marrack, P. and Kappler, J. (1990). The staphylococcal enterotoxins and their relatives. Science 248, 705-11.

37. McCormack, J. E., Callahan, J. E., Kappler, J. and Marrack, P. C. (1993). Profound deletion of mature T cells in vivo by chronic exposure to exogenous superantigen. J. Immunol. 150, 3785-92.

38. McCormack, J. E., Kappler, J. and Marrack, P. (1994). Stimulation with specific antigen can block superantigen-mediated deletion of T cells in vivo. Proc. Natl. Acad. Sci. USA 91, 2086-2090.

39. Muller D. Koller, B. H. W., J. L. LaPan, K. E. Grigman, K. K. Frelinger, J. A. (1992). LCMV-specific, Class II-restricted Cytotoxic T Cells in B₂-Microglobulin deficient Mice. Science 255, 1576-1578.

40. Naor, D., Essery, G., Tarcic, N., Kahan, M. and M., F. (1991). Human Bifunctional T Cell Clones Regulate the Activity of an Autologous T Cell Clone. Ann. NY. Acad. Sci. 636, 135-146.

41. Offner, H., Hashim, G. A. and Vandenbark, A. A. (1991). T cell receptor peptide therapy triggers autoregulation of experimental encephalomyelitis. Science 251, 430-432.

42. Paul, P., Fauchet, R. and Boscher, M. Y. (1987). Isolation of a human major histocompatibility complex class I gene encoding a nonubiquitous molecule expressed on activated lymphocytes. Proc. Nat. Acad. Sci. 84, 2872-6.

43. Rellahan, B. L., Jones, L. A., Kruisbeek, A. M., Fry, A. M. and Matis, L. A. (1990). In vivo induction of anergy in peripheral V beta 8⁺ T cells by staphylococcal enterotoxin B. J. Exp. Med. 172, 1091-100.

44. Salgame, P., Abrams, J. S., Clayberger, C., Goldstein, H., Convit, J., Modlin, R. L. and Bloom, B. R. (1991). Differing lymphokine profiles of functional subsets of human CD4 and CD8 T cell clones. Science 254, 279-82.

45. Shawar, S., M, Vyas, J. M., Rodgers, J. R. and Rich, R. R. (1994). Antigen presentation by major histocompatibility complex class I-B molecules. Ann. Rev. Immunol. 12, 839-80.

46. Shimizu, Y., van Seventer, G. A., Horgan, K. J. and Shaw, S. (1990). Roles of adhesion molecules in T-cell recognition: Fundamental similarities between four integrins on resting human T cells (LFA-1, VLA-4, VIA-5, VLA-6) in expression, binding and costimulation. Immunol. Rev. 114, 109-143.

47. Shinohara, N., Watanabe, M., Sachs, D. H. and Hozumi, N. (1988). Killing of antigen-reactive B cells by class II-restricted, soluble antigen-specific CD8⁺ cytolytic T lymphocytes. Nature 336, 481-4.

48. Stallcup, K. C., Springer, T. A. and Mescher, M. F. (1981). Characterization of an anti-H-2 monoclonal antibody and its use in large-scale antigen purification. J. Immunol. 127, 923-930.

49. Stanton, T. H. and Boyse, E. A. (1979). A new serologically defined locus, Qa-1, in the Tla-region of the mouse. Immunogenetics 3, 525-531.

50. Stanton, T. H., and Carbon, S. (1983). Effect of interferon on Qa-1 expression. Transplantation 36, 352.

51. Staunton, D. E., Marlin, S. D., Stratowa, C., Dustin, M. L. and Springer, T. A. (1988). Primary structure of ICAM-1 demonstrates interaction between members of the immunoglobulin and integrin supergene families. Cell 52, 925-933.

52. Stroynowski, I. (1990). Molecules related to Class I major histocompatibility complex antigens. Ann. Rev. Immunol. 8, 501-530.

53. Sun, D., Qin, Y., Chluba, J., Epplen, J. T. and Wekerle, H. (1988). Suppression of experimentally induced autoimmune encephalomyelitis by cytolytic T-T cell interactions. Nature 332, 843-5.

54. Takahashi, S., Maecker, H. T. and Levy, R. (1989). DNA fragmentation and cell death mediated by T cell antigen receptor/CD3 complex on a leukemia T cell line. Eur. J. Immunol. 19, 1911-1919.

55. Thomas, Y., Sosman, J., Irigoyen, O., Friedman, S. M., Kung, P. C., Goldstein, G. and Chess, L. (1980). Functional analysis of human T cell subsets defined by monoclonal antibodies. I. Collaborative T-T interactions in the immunoregulation of B cell differentiation. J. Immunol. 125, 2402-8.

56. Vandenbark, A. A., Hashim, G. and Offner, H. (1989). Immunization with a synthetic T-cell receptor V-region peptide protects against experimental autoimmune encephalomyelitis. Nature 341, 541-4.

57. Webb, S., Morris, C. and Sprent, J. (1990). Extrathymic tolerance of mature T cells: clonal elimination as a consequence of immunity. Cell 63, 1249-56.

58. Williams, A. F. and Barclay, A. N. (1988). The immunoglobulin superfamily-domains for cell surface recognition. Annu. Rev. Immunol. 6, 381-405.

59. Zhang, J., Medaer, R., Stinissen, P., Hafler, D. and Raus, J. (1993). MHC-restricted depletion of human myelin basic protein-reactive T cells by T cell vaccination. Science 261, 1451-1454.

60. Zijlstra, M., Bix, M., Simister, N. E., Loring, J. M., Raulet, D. H. and Jaenisch, R. (1990). B₂-microglobulin deficient mice lack CD4⁻8⁺ cytolytic T cells. Nature 344, 742-746.

61. Zijlstra, M., Li, E., Sajjadi, F., Subramani, S. and Jaenisch, R. (1989). Germ-line transmission of a disrupted B₂-microglobulin gene produced by homologous recombination in embryonic stem cells. Nature 342, 435-438.

What is claimed is:

1. A method for assaying the level of CD8⁺ T cell cytotoxicity directed toward activated CD4⁺ T cells expressing a specific T cell receptor Vβ chain and a major histocompatibility complex class Ib molecule in a sample, comprising:
a) contacting the sample with the activated CD4⁺ T cells expressing the specific T cell receptor Vβ chain and the major histocompatibility complex class Ib molecule for a determined period of time; and
b) determining the amount of activated CD4⁺ T cell death during the time period, thereby assaying the level of CD8⁺ T cell cytotoxicity directed toward activated CD4⁺ T cells expressing the specific T cell receptor Vβ chain and the major histocompatibility complex class Ib molecule.

2. The method of claim 1, wherein the major histocompatibility complex molecule is murine Qa-1b.

3. The method of claim 1, wherein the major histocompatibility complex mollecule is a non-murine class 1b molecule homologous to murine Qa-1b.

4. The method of claim 1, wherein the sample is a biological sample derived from a subject.

5. The method of claim 4, wherein the biological sample is serum or a tissue sample.

6. The method of claim 4, wherein the subject is a mammal.

7. The method of claim 6, wherein the mammal is a human.

8. The method of claim 6, wherein the mammal is a mouse.

9. The method of claim 1, wherein the activated CD4⁺ T cells in step (a) are labeled with ⁵¹Cr and in step (b) the amount of activated CD4⁺ T cell death is determined by measuring the amount of ⁵¹Cr released from the ⁵¹Cr-labeled activated CD4⁺ T cells.

10. The method of claim 1, wherein the activated CD4⁺ T cells in step (a) are labeled with a fluorescent agent and in step (b) the amount of activated CD4⁺ T cell death is determined by measuring the number of the fluorescently labeled and live activated CD4⁺ T cells by fluorescence associated cell sorter (FACS) analysis.

11. A method for assaying the level of CD8⁺ T cell lymphokine-secreting activity stimulated by activated CD4⁺ T cells expressing a specific T cell receptor Vβchain and a major histocompatibility complex class Ib molecule in a sample, comprising:
a) contacting the sample with the activated CD4⁺ T cells expressing the specific T cell receptor Vβ chain and the major histocompatibility complex class Ib molecule for a determined period of time to stimulate CD8⁺ T cells present in the sample; and
b) determining the amount of a lymphokine released by stimulated CD8⁺ T cells during the time period, thereby assaying the level of CD8⁺ T cell lymphokine-secreting activity stimulated by activated CD4⁺ T cells expressing the specific T cell receptor Vβ chain and the major histocompatibility complex class Ib molecule.

12. The method of claim 11, wherein the major histocompatibility complex molecule is murine Qa-1b.

13. The method of claim 11, wherein the major histocompatibility complex mo ecule is a non-murine class 1b molecule homologous to murine Qa-1b.

14. The method of claim 11, wherein the sample is a biological sample derived from a subject.

15. The method of claim 14, wherein the biological sample is serum or a tissue sample.

16. The method of claim 14, wherein the subject is a mammal.

17. The method of claim 16, wherein the mammal is a human.

18. The method of claim 16, wherein the mammal is a mouse.

19. The method of claim 11, wherein the lymphokine is selected from the group consisting of interleukin-2, γ interferon, and tumor growth factor-beta (TGF-β).

20. The method of claim 11, wherein the amount of lymphokine is determined by radioimmunoassay (RIA), enzyme-linked immunoso rbent assay (ELISA), specific protein mass assay, or activity assay.

21. A method for assaying the level of CD8⁺ T cell activity stimulated by activated CD4⁺ T cells expressing a specific T cell receptor Vβ chain and a major histocompatibility complex class Ib molecule in a sample, comprising:
a) contacting the sample with the activated CD4⁺ T cells expressing the specific T cell receptor Vβ chain and the major histocompatibility complex class Ib molecule for a determined period of time to stimulate CD8⁺ T cells present in the sample; and
b) determining the amount of a cell surface molecule specifically expressed on stimulated CD8⁺ T cells, thereby assaying the level of CD8⁺ T cell activity stimulated by activated CD4⁺ T cells expressing a specific T cell receptor Vβ chain and a major histocompatibility complex class Ib molecule.

22. The method of claim 21, wherein the major histocompatibility complex molecule is murine Qa-1b.

23. The method of claim 21, wherein the major histocompatibility complex molecule is a non-murine class 1b molecule homologous to murine Qa-1b.

24. The method of claim 21, wherein the sample is a biological sample derived from a subject.

25. The method of claim 24, wherein the biological sample is serum or a tissue sample.

26. The method of claim 24, wherein the subject is a mammal.

27. The method of claim 26, wherein the mammal is a human.

28. The method of claim 26, wherein the mammal is a mouse.

29. The method of claim 21, wherein the cell surface molecule specifically expressed on stimulated CD8⁺ T cells is an interleukin-2 receptor.

30. The method of claim 21, wherein/the cell surface molecule specifically expressed on stimulated CD8⁺ T cells is a receptor that recognizes a complex of the major histocompatibility complex class Ib molecule and the Vβ chain or a complex of the binding domains of the major histocompatibility complex class Ib molecule and the Vβ chain.

31. The method of claim 21, wherein the cell surface molecule specifically expressed on stimulated CD8⁺ T cells in step (b) is labeled with a fluorescent agent and the amount of the cell surface receptor is determined/by measuring the intensity of the fluorescently labeled CD8⁺ T cells by fluorescence associated cell sorter (FACS) analysis.

32. A method of suppressing an immune response mediated by activated CD4⁺ T cells expressing a s T cell receptor Vβ chain and a ma stocompatibility complex class Ib molecu n a subject comprising administering to the ect an effective amount of an agent capable imulating CD8⁺ T cell cytotoxicity directed specifically toward the activated CD4⁺ T cells, thereby suppressing the immune response in the subject.

33. The thod of claim 32, wherein the agent is a cell that expresses on the cell surface the specific T cell receptor Vβ chain and the major histocompatibility complex class Ib molecule.

34. The method of claim 32, wherein the agent comprises the major histocompatibility complex lass Ib molecule or a CD8⁺ T cell-binding domain the eof complexed to the Vβ chain or a CD8⁺ T cell-binding domain thereof.

35. The method of claim 32, herein the agent is administered orally, subcutaneously, or intravenously.

36. The method of claim 32, wherein the major histocompatibility complex molecule is murine Qa-1b.

37. The method of claim 32, wherein the major histocompatibility complex molecule is a non-murine class 1b molecule homologous to murine Qa-1b.

38. A method of treating an autoimmune disease comprising the method of claim 2.

39. The method of claim 38, wherein the autoimmune disease is selected from the group consisting of rheumatoid arthritis, multiple sclerosis, scleroderma, systemic lupus erythematosus, idiopathic thrombocytopenia purpura, hemolytic anemia, diabetes, and juvenile diabetes.

40. A method of suppressing an immune response mediated by activated CD4⁺ T cells expressing a specific T cell receptor Vβ chain and a major histocompatibility complex class Ib molecule, in a subject comprising:
a) contacting CD8⁺ T cells with an effective amount of an agent capable of stimulating CD8⁺ T cell cytotoxicity directed specifically toward activated CD4⁺ T cells expressing the specific T cell receptor Vβ chain and the major histocompatibility complex class Ib molecule; and
b) administering to the subject an amount of the stimulated CD8⁺ T cells effective to kill the activated CD4⁺ T cells, thereby suppressing the immune response in the subject.

41. The method of claim 40, wherein the agent is a cell that expresses on the cell surface the specific T cell receptor Vβ chain and the major histocompatibility complex class Ib molecule.

42. The method of claim 40, wherein the agent comprises a complex of the major histocompatibility complex class Ib molecule and the Vβ chain or a complex of the binding domains of the major histocompatibility complex class Ib molecule and the Vβ chain that are recognized by CD8⁺ T cells.

43. The method of claim 40, wherein the stimulated CD8⁺ T cells are administered intravenously.

44. The method of claim 40, wherein the major histocompatibility complex molecule is murine Qa-1b.

45. The method of claim 40, wherein the major histocompatibility complex molecule is a non-murine class 1b molecule homologos to murine Qa-1b.

46. A method of treating an autoimmune disease comprising the method of claim 40.

47. The method of claim 46, wherein the autoimmune disease is selected from the group consisting of rheumatoid arthritis, multiple sclerosis, scleroderma, systemic lupus erythematosus, idio athic thrombocytopenia purpura, hemolytic anemia, diabetes, and juvenile diabetes.

48. A method of suppressing an immune response mediated by activated CD4⁺ T cells expressing a specific T cell receptor Vβ chain and a major histocompatibility complex class Ib molecule in a subject, comprising: administering to the subject an effective amount of an agent capable of inducing expression of the major histocompatibility complex class Ib molecule on the surface of cells that express the T cell receptor Vβ chain, so as to stimulate CD8⁺ T cell cytotoxicity directed specifically toward the activated CD4⁺ T cells thereby suppressing the immune response in the subject.

49. The method of claim 48, wherein the agent is selected from the group consisting of cytokines, interferons, and heat shock proteins.

50. The method of claim 48, wherein the agent is β interferon.

51. The method of claim 48, wherein the agent is administered orally, subcutaneously, or intravenously.

52. The method of claim 48, wherein the major histocompatibility complex molecule is murine Qa-1b.

53. The method of claim 48, wherein the major histocompatibility complex molecule is a non-murine class 1b molecule hom logous to murine Qa-1b.

54. A method of treating an autoimmune disease comprising the method of claim 48.

55. The method of claim 54, wherein the autoimmune disease is selected from the group consisting of rheumatoid arthritis, multiple sclerosis, scleroderma, systemic lupus erythematosus, idiopathic thrombocytopenia purpura, hemolytic anemia, diabetes, and juvenile diabetes.

56. A method of inhibiting the suppression of an immune response mediated by activated CD4⁺ T cells expressing a specific T cell receptor Vβ chain and a major histocompatibility complex class Ib molecule in a subject, comprising: administering to the subject an effective amount of an agent capable of inhibiting the stimulation of CD8⁺ T cell cytotoxicity directed specifically toward the activated CD4⁺ cells by the major histocompatibility complex class Ib molecule and the T cell receptor Vβ chain on the cell surface of activated CD4⁺ cells, thereby inhibiting the suppression of the immune response in the subject.

57. The method of claim 56, wherein the agent is administered orally, subcutaneously, or intravenously.

58. The method of claim 56, wherein the agent is an antibody capable of specifically binding to the major histocompatibility complex molecule.

59. The method of claim 56, wherein the major histocompatibility complex molecule is murine Qa-1b.

60. The method of claim 56, wherein the major histocompatibility complex molecule is a non-murine class 1b molecule homologous to murine Qa-1b.

61. The method of claim 56, wherein the subject is a mammal.

62. The method of claim 61, wherein the mammal is a mouse.

63. A method of treating a disease elected from the group consisting of acquired immunodeficiency syndrome, chronic tuberculosis, chronic leprosy, and chronic tumors comprising the method of claim 56.
